(19) United States
(12) Patent Application Publication
(10) Pub. No.: US 2022/0291011 A1
(43) Pub. Date: Sep. 15, 2022

(54) METHOD AND APPARATUS FOR PROVIDING NAVIGATION AND LOCATION RECOMMENDATION BASED ON GEOSPATIAL VACCINATION DATA

(71) Applicant: HERE Global B.V., Eindhoven (NL)

(72) Inventors: Olga SELINA, Barrington, IL (US); Chris DOUGHERTY, Highland Park, IL (US)

(21) Appl. No.: 17/201,899

(22) Filed: Mar. 15, 2021

(51) Int. Cl. G01C 21/36; H04W 4/024; H04W 4/029; G06F 16/29; G16H 50/80; G16H 50/70; G01C 21/20

(52) U.S. Cl. CPC G01C 21/3694; H04W 4/024; H04W 4/029; G06F 16/29; G16H 50/80; G16H 50/70; G01C 21/3679; G01C 21/206

(57) ABSTRACT

An approach is provided for providing navigation and location recommendation based on geospatial vaccination data. The approach, for example, involves initiating a query of a digital map data for vaccination data associated with a geographic area. The vaccination data indicates a geospatial density of a vaccinated population in the geographic area. The approach also involves processing the vaccination data to determine a navigation route, a recommended point of interest, a recommended event plan, or a combination thereof in the geographic area based on the vaccination data. The approach further involves providing the navigation route, the recommended point of interest, the recommended event plan, or a combination thereof as an output.

### BACKGROUND

As more people are vaccinated and more businesses, workplaces, schools, and points of interest are reopening, people want to know whether a re-opened location has a high or low concentration of unvaccinated individuals that they could be exposed to. The public health authorities around the world have been tracking the spread of contiguous diseases (such as the measles, mumps, flu, HIV, coronavirus (COVID-19), etc.) and the related vaccination rates, to control and prevent the diseases as well as to protect public health and safety. Some public health authorities use surveys and/or reports by vaccine administrators to measure the vaccination rates and provide vaccination data/maps based on the zip codes of vaccinated populations. However, such vaccination data is static and cannot reveal the real-time densities of vaccinated/unvaccinated people at different areas. On the other hand, for example, COVID-19 tracking mobile applications allow an infected individual to voluntarily report via a smart phone, then alert other users that have been nearby the infected individual in the last two weeks to take actions (e.g., getting tested, self-isolating, etc.). Participating in COVID-19 tracking is voluntary thus cannot provide comprehensive tracing data. In addition, such after-exposure approach cannot prevent COVID-19 exposures. Accordingly, service providers and manufacturers face significant technical challenges to assist people to proactively mitigate contiguous disease exposure.

### SOME EXAMPLE EMBODIMENTS

Therefore, there is a need for an approach for providing navigation and location recommendation based on geospatial vaccination data, thereby mitigating exposure to infectious diseases (e.g., COVID-19).

According to one embodiment, a method comprises initiating a query of a digital map data for vaccination data associated with a geographic area. The vaccination data indicates a geospatial density of a vaccinated population in the geographic area. The method also comprises processing the vaccination data to determine a navigation route, a recommended point of interest, a recommended event plan, or a combination thereof in the geographic area based on the vaccination data. The method further comprises providing the navigation route, the recommended point of interest, the recommended event plan, or a combination thereof as an output.

According to another embodiment, an apparatus comprises at least one processor, and at least one memory including computer program code for one or more computer programs, the at least one memory and the computer program code configured to, with the at least one processor, cause, at least in part, the apparatus to monitor a vaccination database for vaccination status data for one or more individuals. The apparatus is also caused to initiate a correlation of the vaccination status data, the one or more individuals, or a combination thereof to one or more locations in a geographic area. The apparatus is further caused to process the correlation to determine a geospatial density of a vaccinated population in the geographic area. The apparatus is further caused to store the geospatial density of the vaccinated population as vaccination data of a geographic database. The apparatus is further caused to provide the geographic database as an output. A navigation route, a recommended point of interest, a recommended event plan, or a combination thereof is determined based on the vaccination data of the geographic database.

According to another embodiment, a computer-readable storage medium carries one or more sequences of one or more instructions which, when executed by one or more processors, cause, at least in part, an apparatus to receive a destination as input. The apparatus is also caused to determine a route from a current location to the destination via a plurality of road segments. One or more of the plurality of road segments to be part of the route is determined based on vaccination data associated with the one or more of the plurality of road segments. The apparatus is further caused to output the determined route or a portion thereof.

According to another embodiment, an apparatus comprises means for initiating a query of a digital map data for vaccination data associated with a geographic area. The vaccination data indicates a geospatial density of a vaccinated population in the geographic area. The apparatus also comprises means for processing the vaccination data to determine a navigation route, a recommended point of interest, a recommended event plan, or a combination thereof in the geographic area based on the vaccination data. The apparatus further comprises means for providing the navigation route, the recommended point of interest, the recommended event plan, or a combination thereof as an output.

In addition, for various example embodiments of the invention, the following is applicable: a method comprising facilitating a processing of and/or processing (1) data and/or (2) information and/or (3) at least one signal, the (1) data and/or (2) information and/or (3) at least one signal based, at least in part, on (or derived at least in part from) any one or any combination of methods (or processes) disclosed in this application as relevant to any embodiment of the invention.

For various example embodiments of the invention, the following is also applicable: a method comprising facilitating access to at least one interface configured to allow access to at least one service, the at least one service configured to perform any one or any combination of network or service provider methods (or processes) disclosed in this application.

For various example embodiments of the invention, the following is also applicable: a method comprising facilitating creating and/or facilitating modifying (1) at least one device user interface element and/or (2) at least one device user interface functionality, the (1) at least one device user interface element and/or (2) at least one device user interface functionality based, at least in part, on data and/or information resulting from one or any combination of methods or processes disclosed in this application as relevant to any embodiment of the invention, and/or at least one signal resulting from one or any combination of methods (or processes) disclosed in this application as relevant to any embodiment of the invention.

For various example embodiments of the invention, the following is also applicable: a method comprising creating and/or modifying (1) at least one device user interface element and/or (2) at least one device user interface functionality, the (1) at least one device user interface element and/or (2) at least one device user interface functionality based on at least in part on data and/or information resulting from one or any combination of methods (or processes) disclosed in this application as relevant to any embodiment of the invention, and/or at least one signal resulting from one or any combination of methods (or processes) disclosed in this application as relevant to any embodiment of the invention.

In various example embodiments, the methods (or processes) can be accomplished on the service provider side or on the mobile device side or in any shared way between service provider and mobile device with actions being performed on both sides.

For various example embodiments, the following is applicable: An apparatus comprising means for performing a method of any of the claims.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 is a diagram of a system capable of providing navigation and location recommendation based on geospatial vaccination data, according to one embodiment;

FIG. 2A is a diagram of an example scenario for selecting a destination and/or routing a vehicle according to a vaccination population map, according to one embodiment;

FIG. 2B is a diagram of an example user interface depicting example vaccination density ranking in an airport, according to one embodiment;

FIG. 3 is a diagram of the components of a disease prevention platform, according to one embodiment;

FIG. 4 is a flowchart of a process for providing navigation and location recommendation based on geospatial vaccination data, according to one embodiment;

FIG. 5 is a flowchart of a process for providing digital vaccination map data thereby providing navigation and location recommendation based on geospatial vaccination data, according to one embodiment;

FIG. 6 is a flowchart of a process for routing based on vaccination data, according to one embodiment;

FIGS. 7A-7D are diagrams of example map user interfaces for providing navigation and location recommendation based on geospatial vaccination data, according to various embodiments;

FIGS. 7E-7G are diagrams of example user interfaces for crowd-sourcing user data with consent, according to various embodiments;

FIG. 8 is a diagram of a geographic database capable of storing map data for providing navigation and location recommendation based on geospatial vaccination data, according to one embodiment;

FIG. 9 is a diagram of hardware that can be used to implement an embodiment;

FIG. 10 is a diagram of a chip set that can be used to implement an embodiment; and

FIG. 11 is a diagram of a mobile terminal (e.g., handset or vehicle or part thereof) that can be used to implement an embodiment.

### DESCRIPTION OF SOME EMBODIMENTS

Examples of a method, apparatus, and computer program for providing underground and/or interior routing or operation of aerial vehicles are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

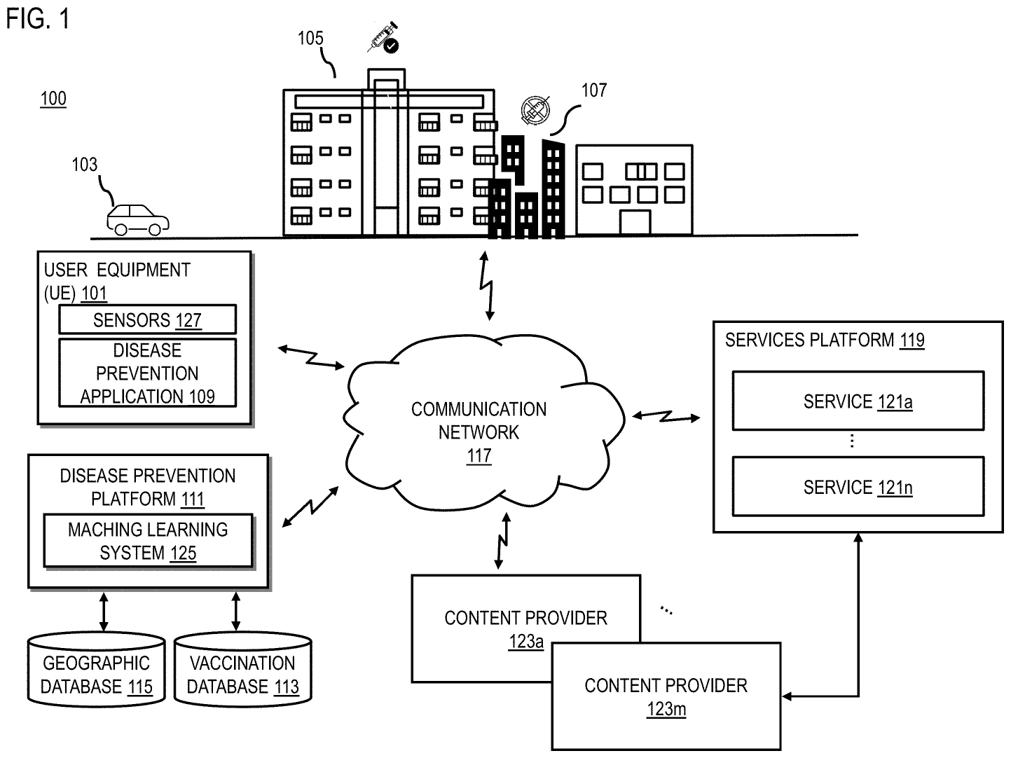

FIG. 1 is a diagram of a system capable of providing navigation and location recommendation based on geospatial vaccination data thereby mitigating exposure to infectious diseases (e.g., COVID-19), according to one embodiment. As noted above, the COVID-19 statistics data (e.g., positive case counts, deaths, vaccination counts, etc.) changes rapidly and differs by location. In terms of vaccination data, the total counts include both confirmed and probable cases in locations identified by public health officials based on criteria developed by government authorities, such as worldwide, per country, per state, pr even per county. For instance, as of Feb. 25, 2021, a total of 21,694,977 doses of COVID-19 vaccine were given to 10,137,747 fully vaccinated people (˜3.1% of population) and 11,453,759 people given at least one dose (˜3.5% of population) in the USA. As a more detailed instance, in Fairfax County, Va., 199,711 vaccine doses have been administered to 71,996 fully vaccinated people (17.4% of population). However, these vaccination data was reported based on people's home addresses that do not reveal their real-time locations for other people to adapt their locations/routes to minimize COVID-19 exposure.

On the other hand, COVID-19 contact tracing applications can identify individuals who may have been in contact with an infected individual using GPS and/or Bluetooth signals to log a user's proximity to other smart phones. Nevertheless, not everyone has a smartphone to record their location data, and not everyone with a smartphone will download a COVID-19 contact tracing application and record their actual vaccination status and location data. In addition, such COVID-19 tracking applications alert users that have been nearby an infected individual in the last two weeks to take actions after exposure, instead of before COVID-19 exposures.

Therefore, user device manufacturers, service providers, operators, etc. face significant technical challenges to assist users to mitigate COVID-19 exposures.

To address these problems, a system 100 of FIG. 1 introduces a capability to assist a user carrying a user equipment (UE) 101 based on vaccination data associated with one or more infectious diseases in a geographic area. Although various embodiments are described with respect to COVID-19, it is contemplated that the approach described herein may be used with other infectious diseases. “Vaccination data can be data that indicates a geospatial density of a vaccinated population associated with one or more targeted infectious diseases in the geographic area. Vaccination data can include one or more markers or indicators associating a person, a location, or a combination thereof with vaccination. Unvaccination data can be data that indicates a geospatial density of an unvaccinated population (e.g., disease-positive, asymptomatic, pre-symptomatic, negative, unknown, etc.) associated with one or more targeted infectious diseases in the geographic area. Unvaccination data can include one or more markers or indicators associating a person, a location, or a combination thereof with unvaccination.

For instance, COVID-19 exposure mitigation can be accomplished via getting close to vaccinated population and/or avoiding unvaccinated population. In one embodiment, the system 100 can determine for a user a location's density of vaccinated and/or unvaccinated individuals, then assist the user to proactively minimize COVID-19 exposure by, for example, visiting locations (e.g., institutions, events, parties, school districts, specific schools, restaurants, malls, stores, etc.) with minimal or no unvaccinated people, routing the user and/or a vehicle 103 carrying the user to a location via routes (e.g., neighborhoods, districts, zones, parks, etc.) with minimal or no unvaccinated people, etc. By way of examples, the system 100 can recommend the user to rent a unit, eat in a restaurant, plan a party, etc. in a building 105 with a high vaccination rate, instead of a building 107 with a high unvaccinated rate (e.g., COVID-19 positive, asymptomatic, anti-vaccine, etc.) to mitigate COVID-19 exposure. By analogy, the system 100 can route the user and or the vehicle 103 to a destination using a route close to the building 105 and away from the building 107 to mitigate COVID-19 exposure.

People remain unvaccinated for many reasons, such as COVID-19 positive, asymptomatic, anti-vaccine, waiting for vaccines, etc., and there is no comprehensive channels to collect unvaccination data. When walking around, most people usually can tell if someone is sick with symptoms (e.g., COVID-19 positive) and naturally avoid them. However, some people can be asymptomatic thus hard to avoid such exposure risk. On the other hand, vaccination data is easier to collect by the public health authorities and/or voluntarily reported by vaccinated individuals thus more reliable for mitigate exposure risk. Therefore, vaccination data is preferred over unvaccination data (including infected population data).

For instance, the vaccination data can be (1) static vaccinated population data retrieved from public health authorities, (2) predicted vaccinated population data estimated based on the static data, vaccinated individual and/or population mobility data, and/or (3) real-time or substantially real-time vaccinated individual and/or population location data. The mobility data of an individual or a population can indicate a history of movements and/or locations of the vaccinated individual or population.

As mentioned, the static data retrieved from public health authorities is based on individual home addresses thus not reflecting dynamic vaccinated and/or unvaccinated population location data. By way of example, the vaccination data can be retrieve from the Centers for Disease Control and Prevention (CDC) of the United States that keeps track of the vaccination rates for the vaccines that are recommended by the Advisory Committee on Immunization Practices (ACIP). ACIP is a group of medical and public health experts who develop recommendations on the use of vaccines to prevent vaccine-preventable diseases (VPDs) that occur in infants, children, adolescents, and adults. CDC uses several surveys to measure vaccination rates for vaccines recommended for different population groups, including COVID-19 vaccination tracking and high level maps for statistical purposes.

In another embodiment, the system 100 can process the static vaccinated and/or unvaccinated population data, individual and/or population mobility data, etc., to predict/simulate dynamic vaccinated and/or unvaccinated population location data in an area, and then assist the user to mitigate COVID-19 exposure via location/POI recommendations and/or routing. For instance, the system 100 can assign an vaccinated or unvaccinated tag as well as an anonymous ID to individual mobility data, then aggregate the individual mobility data into vaccinated or unvaccinated population mobility data for an area by timeline as dynamic vaccinated and/or unvaccinated population spatiotemporal data. This spatiotemporal data can then be used to generate recommended locations/plans and/or routes, and updated based on the anonymous IDs.

Alternatively or concurrently, the system 100 can retrieve real-time or substantially real-time individual and/or population location/mobility data for location/POI recommendations and/or routing. The more current the vaccinated and/or unvaccinated population location data, the more better COVID-19 exposure mitigation. For instance, the system 100 can combine the dynamic vaccinated and/or unvaccinated population spatiotemporal data with real-time vaccinated and/or unvaccinated population spatiotemporal data received from consent individuals, to determine the recommended locations/plans and/or routes. The system 100 can update the location/POI recommendations and/or routing based on updated vaccinated and/or unvaccinated population data.

For example, a user wishing to go out to a restaurant, bar, or store can use the system 100 to find out what POIs near the user has a low concentration of unvaccinated patrons or workers and get suggested disease prevention routing information. As another example, a family can use the system 100 to determine if they should send their children to a school or opt for online classes, or the closest school alternative to mitigate infectious disease exposure. As another example, road trip travelers can also use the system 100 to plan longer segmented routes to avoid making stops at places (e.g., POIs, attractions, towns, etc.) with low levels of vaccination.

In one embodiment, the system 100 can uses an application and/or service platform (e.g., a disease prevention application 109 within the UE 101, a disease prevention platform 111, etc.) of collecting and analyzing vaccination information from a central vaccine tracking database (e.g., a vaccination database 113) that tracks vaccines and drug use, to help a user (e.g., healthy, at risk, or unvaccinated individual) to find locations and/or route where there are minimal or no unvaccinated people, or where the vaccination rate is higher and safer. For example, the system 100 can assist the user to house/apartment hunt and choose a “safer” building, school district, to select a road trip destination/route, to plan an event, etc.

It is contemplated that the route can be generated to mitigate COVID-19 exposure at a ground level. In one embodiment, the COVID-19 exposure is minimized by generating the route to travel via the portions of the indoor/outdoor area where the vaccination densities are greater than a threshold value.

In one embodiment, the system 100 (e.g., via the disease prevention application 109, the disease prevention platform 111, etc.) can retrieve/generate digital vaccination map data (e.g., stored in the vaccination database 113, a geographic database 115, and/or databases of public health authorities). In another embodiment, the system 100 can incorporate existing vaccination survey data (e.g., submitted by individuals via the disease prevention application 109) as a part of the digital vaccination map data. In another embodiment, the system 100 can store and publish the vaccination map data and/or the vaccination-driven routing data as a data layer of a digital map data of the vaccination database 113 and/or the geographic database 115.

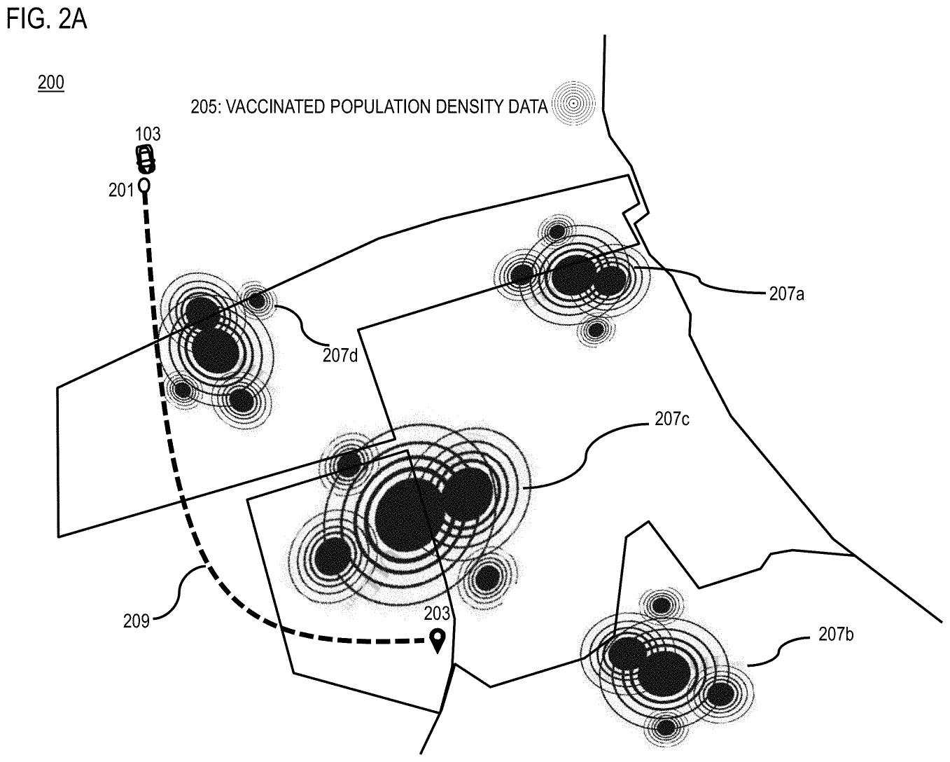

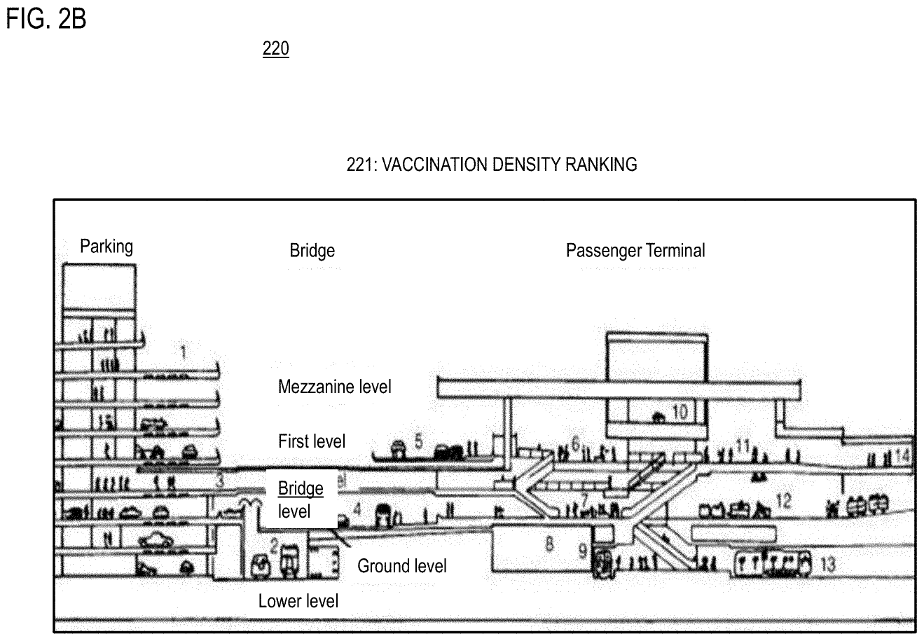

FIG. 2A is a diagram of an example scenario 200 for selecting a destination and/or routing a vehicle according to a vaccination population map, according to one embodiment. In the scenario 200, a user in the vehicle 103 at a current location 201 can select a destination 203 (e.g., an airport) in FIG. 2A bases on an recommendation by the system 100. By way of example, the system 100 can determine that the airport is optimal in term of vaccination rates comparing with all airports in the region, and recommend the user to fly out of the airport.

In one embodiment, instead of taking the shortest/fastest distance route, the system 100 can calculate and recommend a route base on vaccination information (e.g., vaccinated population density data 205 including vaccinated population clusters 207a-207d in the area of interest). In FIG. 2A, the vaccinated population density data 205 is expressed in multiple circles based on the locations and densities. A different shape can be used instead, such as polygons (e.g., a triangle, a square, etc.), irregular polygons, etc. In one embodiment, the vaccinated population density data 205 can be aggregated from three-dimensional (3D) into two-dimensional (2D), such as adding vaccinated individuals in one building into one count of the building associated with the building address. In another embodiment, the vaccinated population density data 205 can be considered in 3D to calculate an optimal route, such as routing via a specific level of a multiple-level highway.

In one embodiment, the system 100 can consider vaccinated population clusters within a threshold distance from a bee line between the current location 201 and the destination 203 (e.g., clusters 207c-207d) to calculate an optimal route. In another embodiment, the system 100 can draw a travel band with a width distance d from the bee line and consider vaccinated population clusters within the travel band to calculate an optimal route. Alternatively or currently, the system 100 can consider vaccinated population clusters meetings a threshold vaccinated population density (e.g., clusters 207b-207d) to calculate an optimal route. The system 100 then can calculate the optimal route (e.g., a route 209) that travels as close to the considered vaccinated population clusters as possible.

In particular, the system 100 can calculate the optimal route at a granular level of road segments or finer as supported by a geographic database (e.g., the geographic database 115 that includes high resolution or high definition (HD) mapping data that provide centimeter-level or better accuracy of map features). By way of example, the route 209 can go through the right side of a segment of a neighborhood road with a row of houses having vaccinated people, and then switching to the left side of another segment of the neighborhood road with another row of houses having vaccinated people. As another example, the system 100 can factor in whether the house windows are open, whether the user stops and/or interacts with a pedestrian on a road segment, etc. to calculate a likelihood of exposure and determine the route 209. Traveling in a zip code with a high vaccination rate does not guarantee zero espouse to infectious diseases, since there are still unvaccinated people. On the other hand, the granular routing supported by the system 100 can specifically mitigate infectious disease exposure by road segment or finer within a zip code regardless of its vaccination rate.

In another embodiment, the system 100 can calculate an optimal mode of transport (e.g., among the vehicle 103, buses, airport shuttles, subways, etc.) to travel to/from the airport as close to the considered vaccinated population clusters as possible, then recommend the user to use the best mode of transport.

In other embodiments, the system 100 can apply the described embodiments to indoor venues (e.g., airport gate waiting areas, hospital waiting rooms, sections of a stadium, etc.) as well, to be close to areas where vaccinated individuals are gathered and/or to avoid areas where unvaccinated individuals are gathered.

Another aspect to reduce the COVID-19 exposure is to consider areas of different altitudes in an indoor open space where air flows can be circulated as risks of COVID-19 exposure, such as department store lobbies, airport terminals, etc. For instance, the COVID-19 exposure can be minimized by generating the route to take a different floor of a building at which the vaccination densities are greater than other floors by a threshold value. This may not always be possible but can be considered by the system 100 to compute a recommended floor for a portion of an indoor route in order to limit the COVID-19 exposure. FIG. 2B is a diagram of an example user interface 220 depicting example vaccination density ranking in an airport, according to one embodiment. FIG. 2B shows fourteen (14) outdoors and indoors locations (e.g., at different levels of a parking area, a bridge area, and a passenger terminal) within an airport that are ranked based on a vaccinated individual count at each location into vaccination density ranking 221, e.g., with “1” being the least vaccinated location/area and “14” being the most vaccinated location/area. In this scenario, the system 100 can calculate an optimal parking spot in the departure/arrival terminal of the airport that is located as close to highly ranked vaccination density location(s) as possible. The system 100 can also calculate an optimal route that travels as close to highly ranked vaccination density location(s) as possible, based on (1) a user current location and a departure/arrival gate, (2) a user current location and a parking spot, (3) a user current location and an optimal mode of transport available at the airport terminal, etc. As the system 10 continuously tracks the movement of vaccinated people in the airport, the system 100 can continuously update the ranking based on the movement of the vaccinated people.

The vaccination population data can be queried over a communication network 117 from vaccination database 113, the geographic database 115 and/or other external sources such as, but not limited, the services platform 119, any of the services 121a-121n (also collectively referred to as services 121, including public health services) of the services platform 119, content providers 123a-123m (also collectively referred to as content providers 123), and/or any other equivalent source. The system 100 can then create vaccination-driven POI data, routing data, event data, etc. based on a digital map for the UE 101. As discussed above, by using vaccination-driven POI recommendation, event planning, routing etc., the system 100 can assist the user to mitigate COVID-10 exposure.

In short, the system 100 can tap into existing database that track vaccination data (e.g., whether individuals have received COVID-19 vaccinations and or booster shots) based on locations, and create dynamic vaccination data based on mobility data and/or real-time location data of vaccinated individuals. The system 100 can anonymize the dynamic vaccination data to support location-based services, such as location recommendation and/or routing in response to user requests for favoring high concentrations of vaccinated individuals and/or avoiding high concentrations of unvaccinated individuals. The system 100 can collect data on routes taken, changing vaccination location patterns, and system use data to provide feedback to the vaccination data sources for further analysis (e.g., to better plan vaccine distribution).

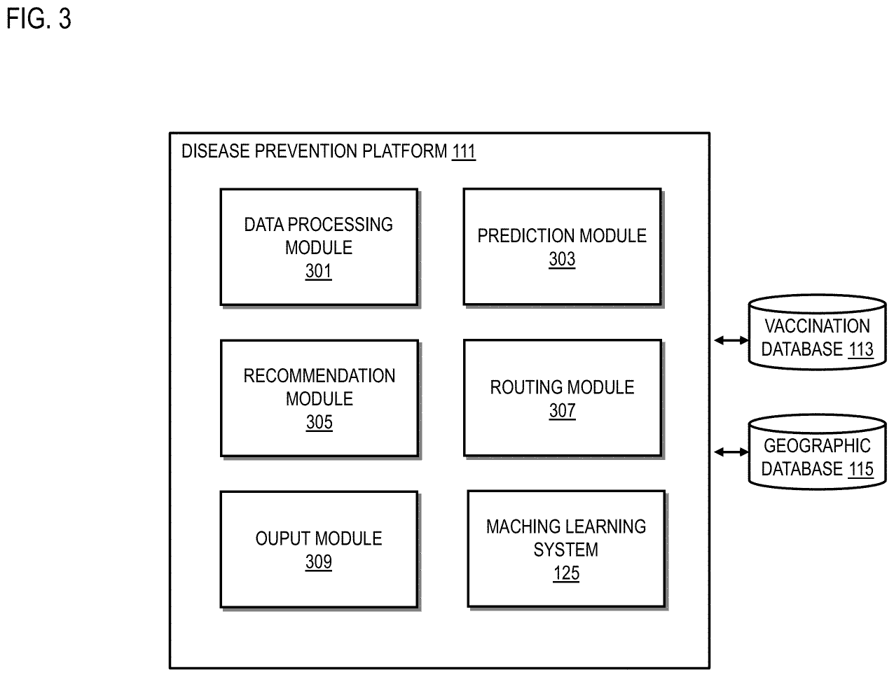

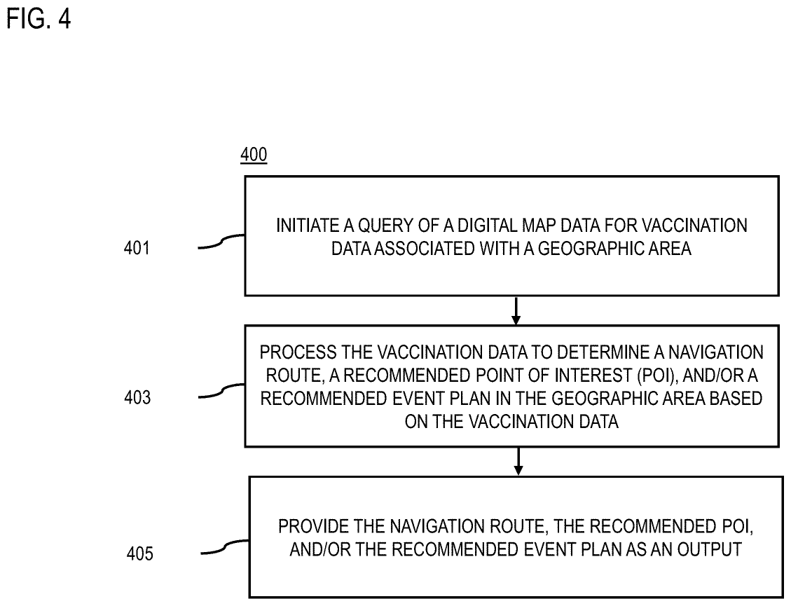

In one embodiment, the disease prevention platform 111 includes one or more components for providing navigation and location recommendation based on geospatial vaccination data, according to the various embodiments described herein. As shown in FIG. 3, the disease prevention platform 111 includes a data processing module 301, a prediction module 303, a recommendation module 305, a routing module 307, and an output module 309. The above presented modules and components of the disease prevention platform 111 can be implemented in hardware, firmware, software, or a combination thereof. It is contemplated that the functions of these components may be combined or performed by other components of equivalent functionality. Though depicted as a separate entity in FIG. 1, it is contemplated that the disease prevention platform 111 may be implemented as a module of any of the components of the system 100 (e.g., a component of the UE 101, the vehicle 103, etc.). In another embodiment, the disease prevention platform 111 and/or one or more of the modules 301-309 may be implemented as a cloud-based service, local service, native application, or combination thereof. The functions of these modules are discussed with respect to FIGS. 4-7 below.

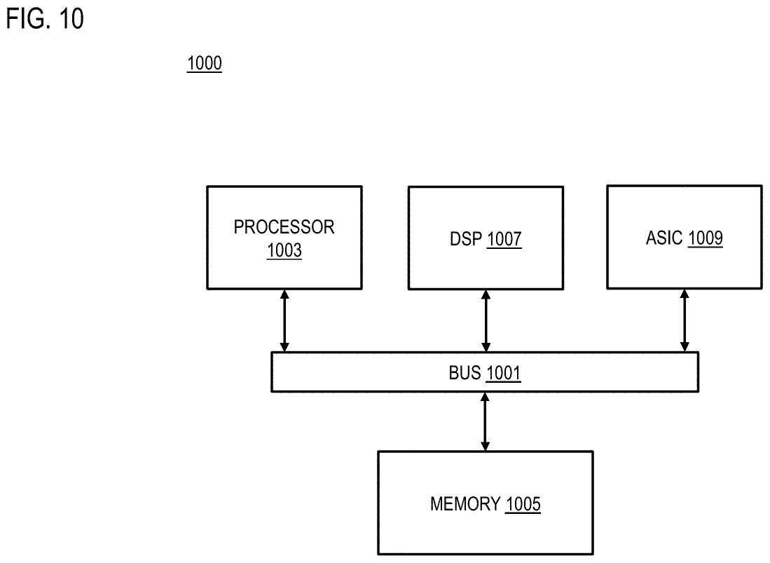

FIG. 4 is a flowchart of a process 400 for providing navigation and location recommendation based on geospatial vaccination data, according to one embodiment. In various embodiments, the disease prevention application 109, the disease prevention platform 111, any of the modules 301-309, and/or a machine learning system 125 of the disease prevention platform 111 may perform one or more portions of the process 400 and may be implemented in, for instance, a chip set including a processor and a memory as shown in FIG. 10. As such, the disease prevention application 109, the disease prevention platform 111, any of the modules 301-309, and/or the machine learning system 125 can provide means for accomplishing various parts of the process 400, as well as means for accomplishing embodiments of other processes described herein in conjunction with other components of the system 100. Although the process 400 is illustrated and described as a sequence of steps, its contemplated that various embodiments of the process 400 may be performed in any order or combination and need not include all of the illustrated steps. More specifically, the process 400 illustrates a process for providing navigation and location recommendation based on geospatial vaccination data thereby mitigating infectious disease exposure.

In one embodiment, for example in step 401, the data processing module 301 can initiate a query of digital map data for vaccination data associated with a geographic area. The vaccination data indicates a geospatial density of a vaccinated population in the geographic area.

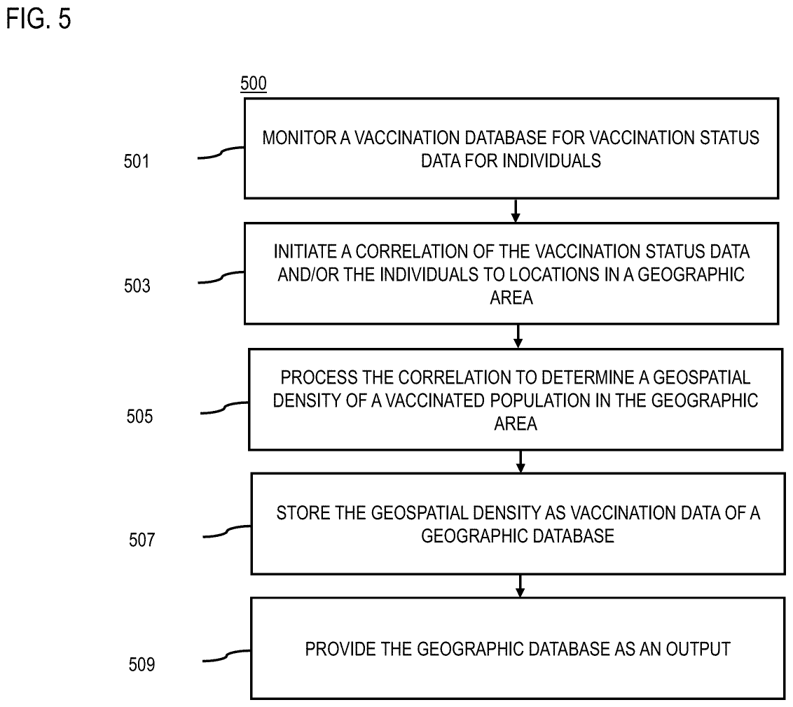

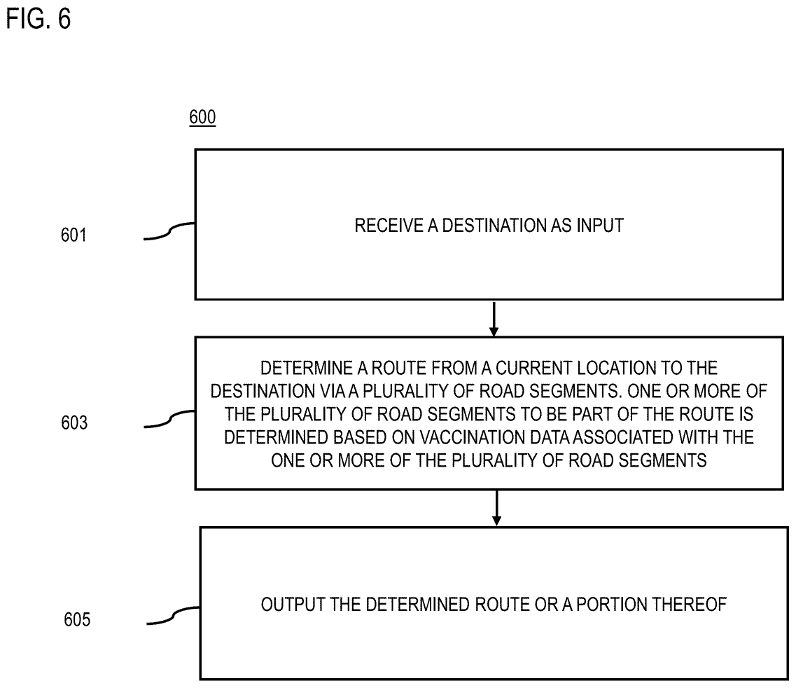

For instance, the vaccination data can be (1) static vaccinated population data retrieved from public health authorities (e.g., CDC), (2) predicted vaccinated population data estimated based on the static data, vaccinated individual and/or population mobility data (e.g., as later generated using a process depicted in FIG. 5), and/or (3) real-time or substantially real-time vaccinated individual and/or population location data (e.g., as submitted by individuals via UE. 101 using a process depicted in FIG. 6). The mobility data of a vaccinated individual or population can indicate a history of movements and/or locations of the vaccinated individual or population.

In one embodiment, in step 403, the recommendation module 305 can process the vaccination data to determine a recommended point of interest (e.g., the destination 203 in FIG. 2A, the airport terminal in FIG. 2B, etc.), a recommended event plan (e.g., a wedding party in Las Vegas), or a combination thereof in the geographic area based on the vaccination data in a vaccination-driven mode and/or a unvaccinated-adverse mode to mitigate COVID-19 exposure. For instance, the vaccination-driven mode can arrange a user to be as close to vaccinated individual/population as possible, while the unvaccinated-adverse mode can arrange a user to be as far away from unvaccinated individual/population as possible.

Alternatively or currently, the routing module 307 can process the vaccination data to determine a navigation route (e.g., the route 209 in FIG. 2A) in the geographic area based on the vaccination data (e.g., in the vaccination-driven mode and/or the unvaccinated-adverse mode to mitigate COVID-19 exposure).

In one embodiment, the data processing module 301 can receive a request specifying or providing data for specifying a threshold density of the vaccinated population (e.g., 35% of population per square miles). By way of example, the threshold density can be set by public health authorities, the user, and/or the data processing module 301, arbitrarily or based on factors such as health status, medical conditions, medication conditions, etc. of the user, the public, etc. The navigation route, the recommended point of interest, the recommended event plan, or a combination thereof can then be determined by the routing model 307 or the recommendation module 305 further based on the threshold density.

By way of examples, the navigation route, the recommended point of interest, the recommended event plan, or a combination thereof can determined based favoring or avoiding one or more portions of the geographic area associated with vaccination data indicating that the vaccinated population is above or below the threshold density. For instance, the data for specifying the threshold density can include user medical condition data regarding at least one user associated with the request, and the threshold density can determined based on the user medical condition data (e.g., including a vaccination status of the at least one user). More specifically, the user medical condition data can include (1) all diseases, lesions, disorders, or non-pathologic conditions that normally receive medical treatment, such as pregnancy or childbirth, of a user, as well as (2) medication condition data of the user (e.g., vaccination status data of the user).

In other instances, the request can further specify a travel time, a travel distance, or a combination thereof, and the navigation route, the recommended point of interest, the recommended event plan, or a combination thereof can be determined by the routing model 307 or the recommendation module 305 further based on the travel time, the travel distance, or a combination thereof. For instance, the request can specify a work lunch within walking distance, and the recommendation module 305 can recommend a restaurant nearby work with a clientele of a high vaccination rate (meeting the threshold density) during weekday lunch time. In addition, the recommendation module 305 can recommend an optimal route (meeting the threshold density) to the restaurant during weekday lunch time.

In another embodiment, the vaccinated population can include a working population working at the recommended point of interest (e.g., staffs working in the restaurant), at a location (e.g., a hotel) associated with the recommended event plan (e.g., the wedding party in Las Vegas), within a threshold distance of the navigation route (e.g., a walking path to the restaurant, a driving route to the hotel, etc.), or a combination thereof.

In yet another embodiment, the prediction module 303 can monitor one or more real-time parameters (e.g., changed public transport schedules, unexpected weather conditions, traffic accidents, protests, etc.) that affect the geospatial density of the vaccinated population, for the routing model 307 to update the navigation route and/or for the recommendation module 305 to update the recommended point of interest, the recommended event plan, or a combination thereof based on the one or more real-time parameters.

In another embodiment, the navigation route, the recommended point of interest, the recommended event plan, or a combination thereof relates to indoor navigation or indoor mapping (e.g., the airport terminal in FIG. 2B).

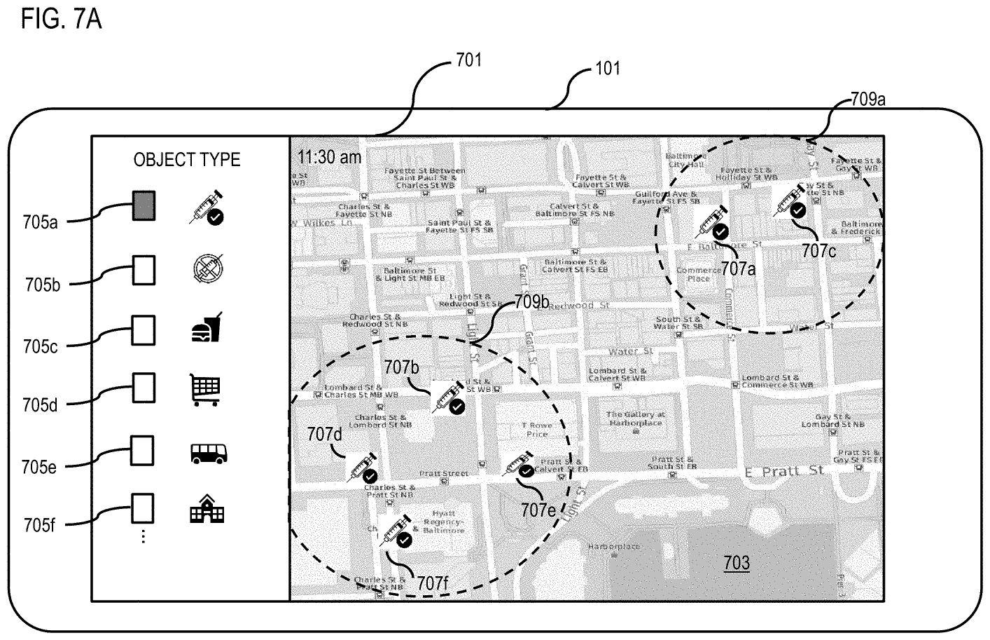

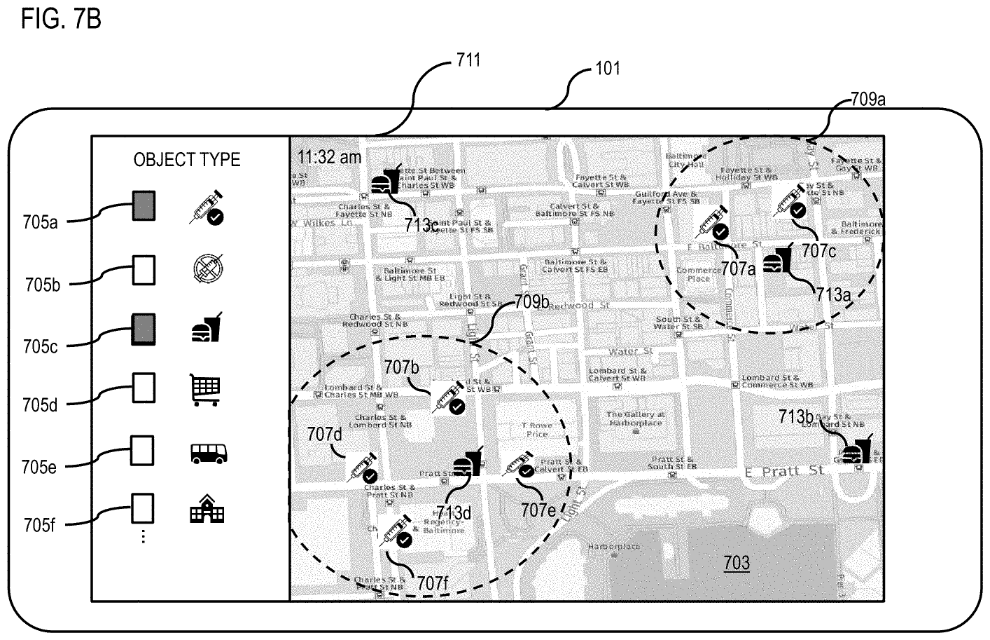

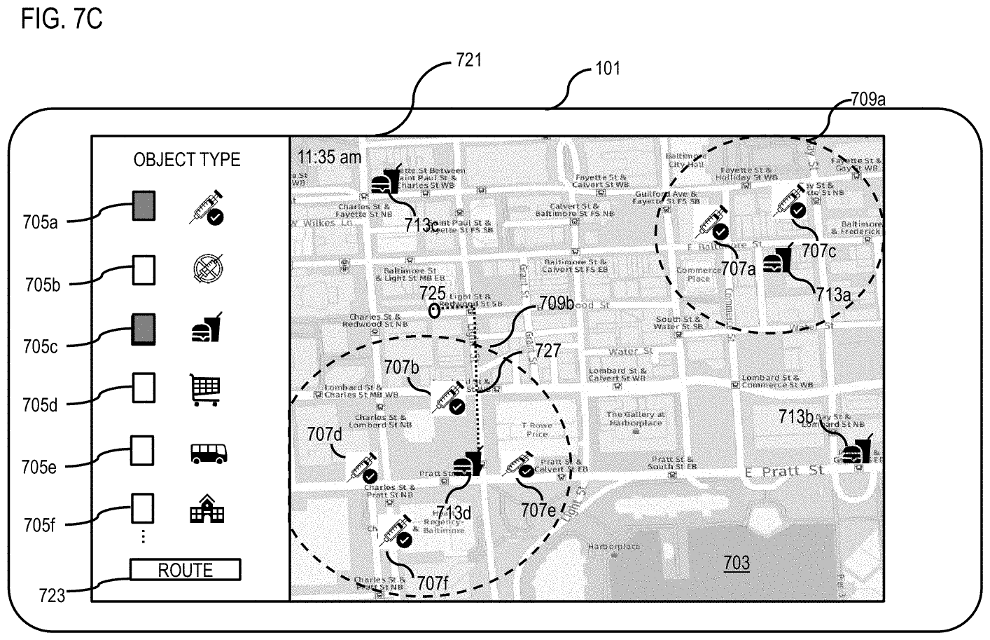

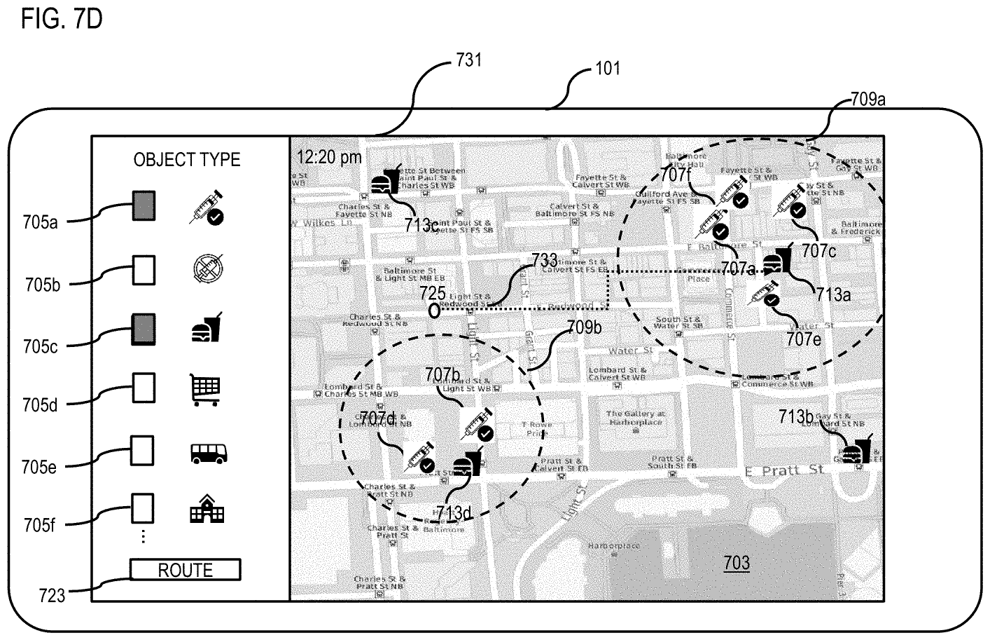

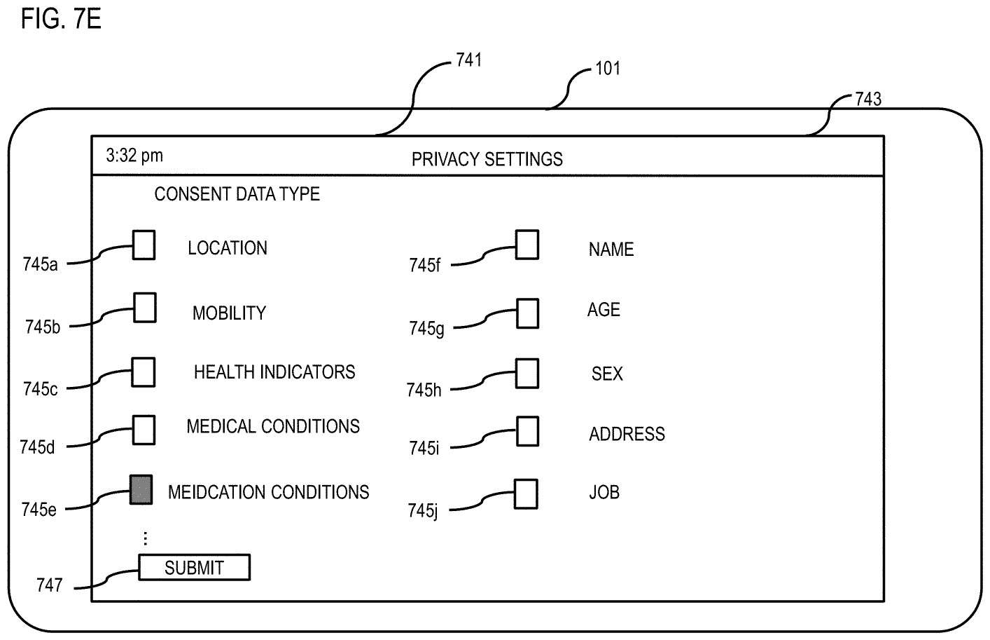

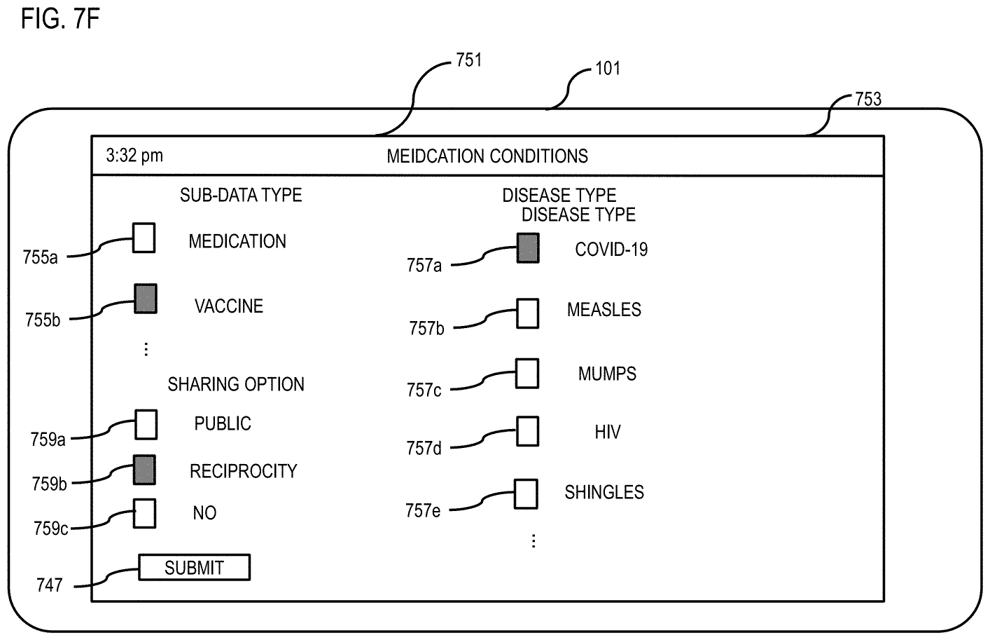

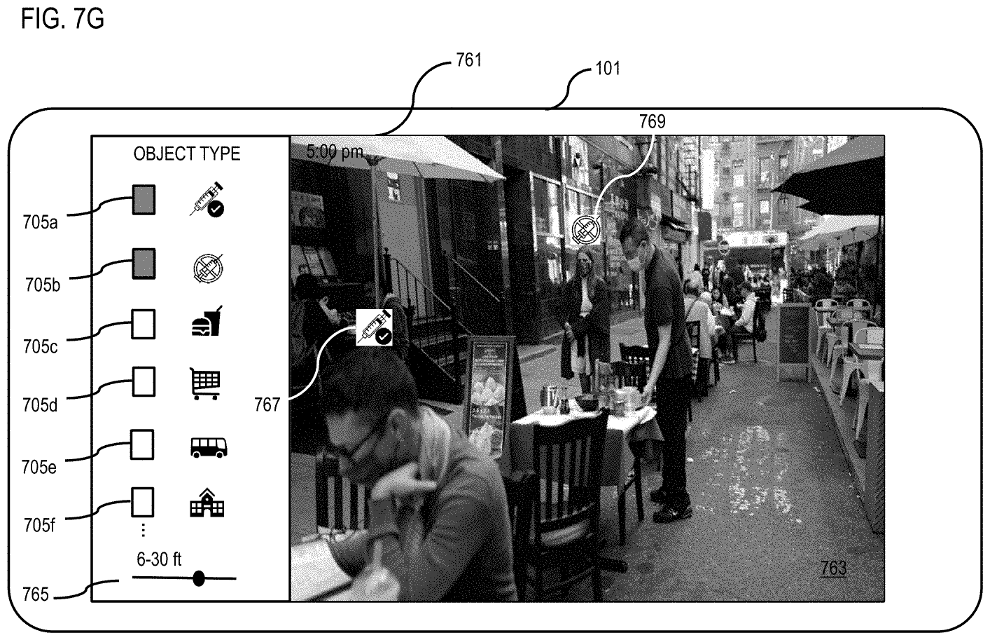

In one embodiment, in step 405, the output module 309 can provide the navigation route, the recommended point of interest, the recommended event plan, or a combination thereof as an output. In another embodiment, the output module 309 can process the output to generate a map user interface presenting a real-time map representation of the vaccination data with respect to the geographic area, the navigation route, the recommended point of interest, the recommended event plan, or a combination. FIGS. 7A-7D are diagrams of example map user interfaces for providing navigation and location recommendation based on geospatial vaccination data, according to various embodiments.

Referring to FIG. 7A, in one embodiment, the system 100 can generate a user interface (UI) 701 (e.g., the disease prevention application 109) for a UE 101 (e.g., a mobile device, a smartphone, a client terminal, etc.) that can allow a user to see vaccination population density currently and/or over time (e.g., an hour, a day, a week, a month, a year, etc.) in an area, where static vaccination data (e.g., from public health authorities) and/or dynamic vaccination data (e.g., predicted and/or crowd-sourced by the system 100) is available in digital map data, to be presented via a map 703 upon selection of one or more object types. For instance, the object types in FIG. 7A includes vaccinated people/population 705a, unvaccinated people/population 705b, restaurant 705c, supermarket 705d, bus stop 705e, school 705f, etc.

In FIG. 7A, for example, in response to a user selection of the vaccinated people/population 705a at 11:30 am, and the system 100 can determine and present in the map 703 six groups of vaccinated people/populations 707a-707f (e.g., working in different entities and received vaccines via work) that make into two vaccinated clusters 709a, 709b. In FIG. 7B, in response to another user selection of the restaurant data object 705c at 11:32 am, the system 100 can generate an UI 711 showing four restaurants 713a-713d in the map 703.

Referring to FIG. 7C, in one embodiment, the system 100 can generate an UI 721 showing a “route” button 723. In response to another user selection of the “route” button 723 at 11:35 am, the UI 721 can also show a current user location 725, and a route 727 leading to the restaurant 713d which is determined by the system 100 as the optimal restaurant within walking distance and with most exposure to vaccinated people among the four restaurants 713a-713d in the map 703.

However, the user was delayed by work then check again with the system 100 at 12:20 pm in FIG. 7D. By this time, a lot of people (including vaccinated individuals) have left their offices and arrived at restaurants 713a, 713d. In response to another user selection of the “route” button 723 at 12:20 pm, the UI 731 can show the current user location 725, and a new route 733 leading to the restaurant 713a which is determined by the system 100 as the optimal restaurant within walking distance and with most exposure to vaccinated people among the four restaurants 713a-713d in the map 703 at 12:20 pm.

FIG. 5 is a flowchart of a process 500 for providing digital vaccination map data thereby providing navigation and location recommendation based on geospatial vaccination data, according to one embodiment. In various embodiments, the disease prevention application 109, the disease prevention platform 111, any of the modules 301-309, and/or a machine learning system 125 of the disease prevention platform 111 may perform one or more portions of the process 500 and may be implemented in, for instance, a chip set including a processor and a memory as shown in FIG. 10. As such, the disease prevention application 109, the disease prevention platform 111, any of the modules 301-309, and/or the machine learning system 125 can provide means for accomplishing various parts of the process 500, as well as means for accomplishing embodiments of other processes described herein in conjunction with other components of the system 100. Although the process 500 is illustrated and described as a sequence of steps, its contemplated that various embodiments of the process 500 may be performed in any order or combination and need not include all of the illustrated steps. More specifically, the process 500 illustrates a process for creating and storing digital map representing vaccination data thereby recommending POIs, planning events, determining routes, etc.

In one embodiment, for example in step 501, the data processing module 301 can monitor a vaccination database (e.g., the vaccination database 113) for vaccination status data for one or more individuals. By way of example, the data processing module 301 can access the vaccination database 113 constantly, and the vaccination status data (e.g., ID, location, movement, timeframe, etc.) would be processed and updated in real-time or substantially real-time.

In one embodiment, in step 503, the prediction module 303 can initiate a correlation of the vaccination status data, the one or more individuals (e.g., vaccinated population clusters 207a-207d in FIG. 2A), or a combination thereof to one or more locations in a geographic area (e.g., the area depicted in FIG. 2A, the area depicted in the map 703 of FIG. 7A, etc.)

In one embodiment, in step 505, the prediction module 303 can process the correlation to determine a geospatial density (e.g., expressed in multiple circles in FIG. 2A) of a vaccinated population (e.g., any one of the vaccinated population clusters 207a-207d in FIG. 2A) in the geographic area.

In one embodiment, the prediction module 303 can anonymize the vaccination status data for the one or more individuals. For instance, the prediction module 303 can run the vaccination status data through a scrubber to remove any identification data associated with the one or more individuals. As such, the vaccination data of the geographic database (e.g., the vaccination database 113) can be based on the anonymized vaccination status data (e.g., to maintain individual privacy).

In one embodiment, the prediction module 303 can collect mobility data (e.g., including historical and/or real-time mobility data) from one or more devices (e.g., UEs 101) associated with the one or more individuals, and the geospatial density of the vaccinated population can be determined further based on the mobility data. For instance, the prediction module 303 can assign an vaccinated tag and an anonymous ID to each individual mobility data, then aggregate individual mobility data into vaccinated population mobility data for an area by timeline to generate dynamic vaccinated population spatiotemporal data. This anonymous spatiotemporal data can then be used to generate recommended locations/plans and/or routes, and updated based on an anonymous IDs.

In one embodiment, the vaccination status data, the mobility data, or a combination thereof can be crowd-sourced from the one or more individuals who consent to collecting the vaccination status data, the mobility data, or combination thereof. FIGS. 7E-7G are diagrams of example user interfaces for crowd-sourcing user data with consent, according to various embodiments.

Referring to FIG. 7E, in one embodiment, the system 100 can generate an UI 741 showing a privacy setting panel 743 listing a lot of consent data types. In FIG. 7E, for example, the listing of consent data types includes location 745a, mobility 745b, health indicators(s) 745c, medical condition(s) 745d, medication condition(s) 745e, name 745f, age 745g, sex 745h, address 745i, job 745j, etc.

The mobility data 745b of a user can indicate a history of user movements and/or locations). For instance, the system 100 can retrieves user historical mobility data from user device sensors 127, vehicle data (e.g., vehicle historical mobility data and/or real-time information), etc., and builds a user mobility pattern model and/or matrix. By way of example, the insights may include when and where the user travel to a location, and the used mode(s) of transport; when and where each mode of transport is released; how long the user stays at a given location; where the user is located within the threshold proximity to a point of interest (e.g., restaurant, supermarket, park, etc.) at a given time; correlations that can be made relative to other factors such as weather, events, day of the week, etc.

The health indicators(s) 745c of a user can indicate access to health services, clinical preventive services, environmental quality, injury and violence, mental health, nutrition, physical activities, etc. The medical condition(s) 745d of a user can include all diseases, lesions, disorders, or non-pathologic conditions that normally receive medical treatment, such as pregnancy or childbirth. The medication condition(s) 745e of a user can include any a substance used for medical treatment, especially a medicine/drug, vaccine, etc.

In response to a user selection of the medication condition(s) 745e and a “submit” button 747 in FIG. 7E, the system 100 can retrieve and present in an UI 751 in FIG. 7F: sub-data types, associated disease types, and a sharing option of the medication condition(s) 745e in a panel 753. For instance, the panel 753 shows sub-data types of medication 755a, vaccine 755b, etc., disease types COVID-19 757a, measles 757b, mumps 757c, HIV 757d, shingles 757e, etc., and a sharing option as public 759a, reciprocity 759b, no 759c. For instance, the use selects to share the medication condition of COVID-19 vaccine (e.g., vaccinated) based on the reciprocity 759b (e.g., only users sharing their corresponding medication condition can access my medication condition of COVID-19 vaccine), by checking the relevant boxes and the “submit” button 747 in FIG. 7F.

Referring FIG. 7G, when the user walks on a street toward a restaurant (e.g., the restaurant 713a in FIG. 7D), the system 100 can generate an UI 761 showing a live street view 763, the object types 705a-705f, and a display range slide bar 765 (e.g., 6-30 feet). In response to user selections of the object types vaccinated people/population 705a and unvaccinated people/population 705b, and a distance of 20 feet, the system 100 can retrieve the COVID-19 vaccination data of users consent to sharing from the vaccination database 113, the geographic database 115, etc. The system 100 then can augment a vaccinated tag 767 for a user A sitting on a dining table, and a unvaccinated tag 769 for a user B walking in the opposite direction into the live street view 763, using augmented reality (AR).

For instance, the vaccinated user A consented to be seen with the vaccinated tag 767, for example, to see other users' vaccination status tags, to promote vaccination, to show off, etc. On the other hand, the unvaccinated user B consented to be seen with the unvaccinated tag 769, for example, to see other users' vaccination status tags, to keep other users away, etc.

There is no tag showing for the waiter may be due to his privacy setting as no sharing, or the waiter does not subscribe to the system 100. As the user continues walking, more tags can surface for other users down the street.

In one embodiment, in step 507, the prediction module 303 can store the geospatial density of the vaccinated population as vaccination data of a geographic database (e.g., the geographic database 115).

In one embodiment, in step 509, the output module 309 can provide the geographic database as an output. A navigation route, a recommended point of interest, a recommended event plan, or a combination thereof can be determined based on the vaccination data of the geographic database via, for example, the process 400. In one embodiment, the output module 309 can provide access to the geographic database to at least one edge device (e.g., UE 101, an onboard nav system of the vehicle 103, etc.), and the edge device can submit at least one request to generate the navigation route, the recommended point of interest, the recommended event plan, or a combination thereof

In one embodiment, a vaccinated population mobility machine learning model can be built by the machine learning system 125 based on vaccinated individual mobility data, context data (e.g., vaccinated individual schedule data, events, traffic, weather, etc.), etc. as training data. By way of example, the machine learning system 125 can determine presence of vaccinated individuals at a location (e.g., POI) using parameters/factors such as characteristics of the user (e.g., preferences, schedules, etc.), characteristics of modes of transport (e.g., model, schedule, mobility records, etc.), traveling context and conditions (e.g., traffic, weather, etc.), map data, etc. that describe a distribution or a set of density distributions of vaccinated population, thereby providing navigation and location recommendation based on geospatial vaccination data, as well as sharing the predicted/estimated vaccination data with various vaccination data sources, such as government/municipality agencies, local or community agencies (e.g., a police department), and/or third-party official/semi-official sources.

In one embodiment, the machine learning system 125 can select respective weights of the parameters/factors, and/or various vaccination data sources, for example, based on their respective reliability. In another embodiment, the machine learning system 125 can further select or assign respective correlations, relationships, etc. among the vaccination data sources, for determining a confidence level of one or more vaccination data points. In one instance, the machine learning system 125 can continuously provide and/or update the vaccinated population mobility machine learning model using, for instance, a support vector machine (SVM), neural network, decision tree, etc.

FIG. 6 is a flowchart of a process 600 for routing based on vaccination data, according to one embodiment. In various embodiments, the disease prevention application 109, the disease prevention platform 111, any of the modules 301-309, and/or a machine learning system 125 of the disease prevention platform 111 may perform one or more portions of the process 600 and may be implemented in, for instance, a chip set including a processor and a memory as shown in FIG. 10. As such, the disease prevention application 109, the disease prevention platform 111, any of the modules 301-309, and/or the machine learning system 125 can provide means for accomplishing various parts of the process 600, as well as means for accomplishing embodiments of other processes described herein in conjunction with other components of the system 100. Although the process 600 is illustrated and described as a sequence of steps, its contemplated that various embodiments of the process 600 may be performed in any order or combination and need not include all of the illustrated steps. More specifically, the process 600 illustrates a process for rendering recommending POIs, planning events, determining routes, etc., as well as sharing user vaccination status data and/or user location data.

In one embodiment, for example in step 601, the data processing module 301 can receive a destination as input.

In one embodiment, in step 603, the routing module 307 can determine a route from a current location to the destination via a plurality of road segments. One or more of the plurality of road segments to be part of the route is determined based on vaccination data associated with the one or more of the plurality of road segments (e.g., FIGS. 7C-7D). For instance, the vaccination data can include one or more markers or indicators associating a person, a location, or a combination thereof with vaccination.

In one embodiment, the data processing module 301 can receive, via the user interface, a request specifying a threshold density of the vaccinated data (e.g., 35% of population per square miles), and the determined route or the portion thereof can be determined further based on the threshold density.

In another embodiment, the data processing module 301 can receive, at the device (e.g., UE 101), digital map data including the vaccination data, and the determined route or the portion thereof is determined based favoring or avoiding the plurality of road segments associated with vaccination data indicating that vaccinated population is above or below the threshold density.

In yet another embodiment, the data processing module 301 can receive, via the user interface, a user consent to share user vaccination status data, user mobility data, or a combination thereof (e.g., via the process depicted in FIGS. 7F-7G), and the vaccination data, the determined route or the portion thereof, or a combination thereof is updated further based on the user vaccination status data, the user mobility data, or a combination thereof.

In one embodiment, in step 605, the output module 309 can output the determined route or a portion thereof.

Users can subscribe to the system 100 and access vaccination data. In one embodiment, for example, the data processing module 301 can receive a query via an user interface of a device for a navigation route, a recommended point of interest, a recommended event plan, or a combination thereof. The navigation route, the recommended point of interest, the recommended event plan, or a combination thereof is generated based on vaccination data that indicates a geospatial density of a vaccinated population in a geographic area.

For instance, the vaccination data can be real-time or substantially real-time vaccinated individual and/or population location data (e.g., as submitted by individuals via UEs 101). The mobility data of a vaccinated individual or population can indicate a history of movements and/or locations of the vaccinated individual or population.

In one embodiment, the data processing module 301 can receive, via the user interface, a request specifying a threshold density of the vaccinated population (e.g., 35% of population per square miles), and the navigation route, the recommended point of interest, the recommended event plan, or a combination thereof can be determined the routing model 307 or the recommendation module 305 further based on the threshold density.

In one embodiment, the data processing module 301 can receive, at the device (e.g., UE 101), digital map data including the vaccination data. As such, the navigation route, the recommended point of interest, the recommended event plan, or a combination thereof can be determined the routing model 307 or the recommendation module 305 based favoring or avoiding one or more portions of the geographic area associated with vaccination data indicating that the vaccinated population is above or below the threshold density.

In one embodiment, the data processing module 301 can receive, via the user interface, a user consent to share user vaccination status data, user mobility data, or a combination thereof (e.g., via the process depicted in FIGS. 7F-7G). As results, the vaccination data, the navigation route, the recommended point of interest, the recommended event plan, or a combination thereof can be updated by the routing model 307, the recommendation module 305, or the output module 309 further based on the user vaccination status data, the user mobility data, or a combination thereof.

In one embodiment, the output module 309 can render on the user interface the navigation route, the recommended point of interest, the recommended event plan, or a combination thereof (e.g., FIGS. 7C-7D).

In another embodiment, the data output module 309 can provide the user vaccination status data, the user mobility data, or a combination thereof as an output. Then after, the digital map data, the vaccination data, the vaccinated population, or a combination thereof is updated by the disease prevention application 109 and/or the disease prevention platform 111 based on the output.

In another embodiment, the data output module 309 can promote vaccination targeted at the one or more devices based on the unvaccinated status information. For instance, the data output module 309 can start targeted campaigns to specific areas that have high concentrations of unvaccinated individuals.

The above-discussed embodiments combine different technologies (e.g., sensors, 2D/3D routing, vaccination population mapping, dynamic vaccination population mobility modeling, probability computation, risk computation, machine learning, big data analysis, etc.) to mitigate COVID-19 exposure by providing navigation and location recommendation based on geospatial vaccination data.

Returning to FIG. 1, as shown, the system 100 comprises an UE 101 equipped with a variety of the sensors 127 that is capable operating in a vaccination-driven mode, and/or a unvaccinated-adverse mode to mitigate infectious disease exposure. In one embodiment, the UE 101 can provide navigation and location recommendation based on geospatial vaccination data using the disease prevention applications 109 according to the embodiments described herein. As previously discussed, the system 100 further includes the disease prevention platform 111 coupled to the vaccination database 113 and/or the geographic database 115, wherein the disease prevention platform 111 is performs the functions associated with navigation and location recommendation based on geospatial vaccination data as discussed with respect to the various embodiments described herein. In one embodiment, the UE 101, disease prevention platform 111, and other components of the system 100 have connectivity to each other via the communication network 117.

In one embodiment, the vehicle 103 is an autonomous vehicle. In one embodiment, the vehicle 103 is configured to travel using one or more modes of operation for providing navigation and location recommendation based on geospatial vaccination data. The vehicle 103 may include any number of sensors including cameras, recording devices, communication devices, etc. By way example, the sensors may include, but are not limited to, a global positioning system (GPS) sensor for gathering location data based on signals from a positioning satellite, Light Detection And Ranging (LIDAR) for gathering distance data and/or generating depth maps, a network detection sensor for detecting wireless signals or receivers for different short-range communications (e.g., Bluetooth®, Wireless Fidelity (Wi-Fi), Li-Fi, Near Field Communication (NFC), etc.), temporal information sensors, a camera/imaging sensor for gathering image data, and the like. The vehicle 103 may also include recording devices for recording, storing, and/or streaming sensor and/or other telemetry data to the UE 101 and/or the disease prevention platform 111 for providing navigation and location recommendation based on geospatial vaccination data.

In addition, the vehicle 103 can be configured to observe restricted paths or routes. For example, the restricted paths may be based on governmental regulations. In one embodiment, the system 100 may also take into account one or more pertinent environmental or weather conditions (e.g., rain, water levels, sheer winds, etc. in and around underground passageways and their entry/exit points) in determining a navigation route.

In one embodiment, the vehicle 103 may determine contextual information such as wind and weather conditions in route that may affect the vehicle's ability to follow the specified navigation route to pass close to one or more vaccinated population cluster (e.g., using one or more onboard sensors) and then relay this information in substantially real-time to the system 100 for validation. In one embodiment, the vehicle 103 may request one or more modifications of the route based, at least in part, on the determination of the contextual information or a change in the real-time parameters (e.g., dynamic features such as changed public transport schedules, unexpected weather conditions, etc.). In one embodiment, the system 100 creates a data object to represent the navigation route and may automatically modify the route data object based on receipt of the contextual information from the UE 101, the vehicle 103, or another source and then transmit the new route object to the UE 101 and/or the vehicle 103 for execution. In one embodiment, the UE 101 and/or the vehicle 103 can determine or access the new route data object and/or determine or access just the relevant route segments and adjust its current path accordingly.

By way of example, a UE 101 is any type of mobile terminal, fixed terminal, dedicated vehicle control unit, or portable terminal including a mobile handset, station, unit, device, multimedia computer, multimedia tablet, Internet node, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, or any combination thereof, including the accessories and peripherals of these devices, or any combination thereof. It is also contemplated that a UE 101 can support any type of interface to the user (such as “wearable” circuitry, etc.). In one embodiment, a UE 101 may support any type of interface for routing the user and/or the vehicle 103. In addition, a UE 101 may facilitate various input means for receiving and generating information, including, but not restricted to, a touch screen capability, a keyboard and keypad data entry, a voice-based input mechanism, and the like. Any known and future implementations of a UE 101 may also be applicable.

By way of example, the UE 101 may execute the disease prevention application 109, a location-based service application, a navigation application, a content provisioning application, a camera/imaging application, a media player application, an e-commerce application, a social networking application, and/or the like. In one embodiment, the applications may include one or more feature recognition applications used for identifying or mapping features or routes according to the embodiments described herein. In one embodiment, the disease prevention application 109 may act as a client for the disease prevention platform 111 and perform one or more functions of the disease prevention platform 111. In one embodiment, the disease prevention application 109 may be considered as a Graphical User Interface (GUI) that can provide navigation and location recommendation based on geospatial vaccination data, according to the embodiments described herein.

In one embodiment, the communication network 117 of the system 100 includes one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, 5G networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof.0

In one embodiment, the disease prevention platform 111 can interact with the services platform 119 to receive data for providing navigation and location recommendation based on geospatial vaccination data. By way of example, the services platform 119 may include one or more services 121 for providing content, provisioning services, application services, storage services, mapping services, navigation services, contextual information determination services, location-based services, information-based services (e.g., weather), etc. By way of example, the services 121 may provide or store traffic schedule data (e.g., train/subway schedules, elevator schedules, etc.), weather data, and/or other data used by the embodiments describe herein. In one embodiment, the services platform 119 may interact with the UE 101, the vehicle 103, and/or disease prevention platform 111 to supplement or aid in providing navigation and location recommendation based on geospatial vaccination data.

By way of example, the UE 101, the vehicle 103, the disease prevention platform 111, and the services platform 119 communicate with each other and other components of the system 100 using well known, new or still developing protocols. In this context, a protocol includes a set of rules defining how the network nodes within the system 100 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers as defined by the OSI Reference Model.

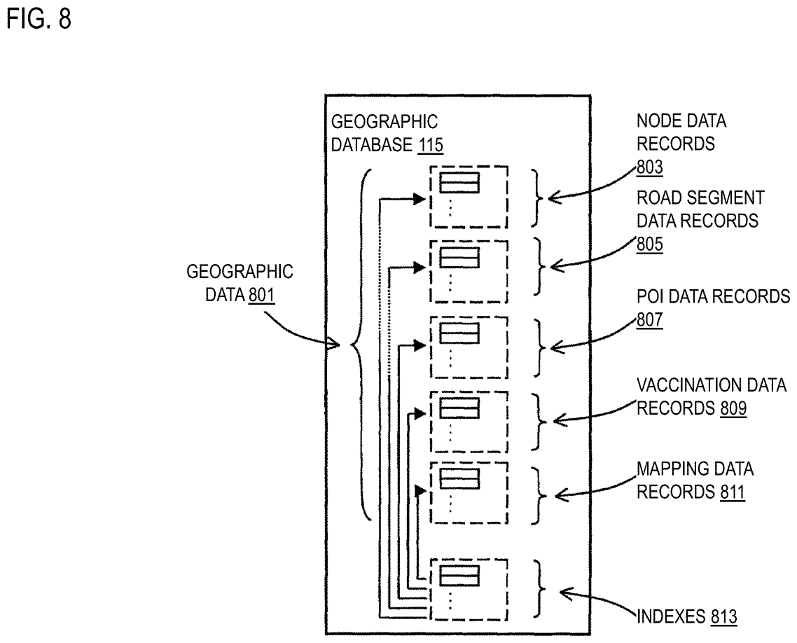

FIG. 8 is a diagram of a geographic database (such as the database 115), according to one embodiment. In one embodiment, the geographic database 115 includes geographic data 801 used for (or configured to be compiled to be used for) mapping and/or navigation-related services, such as for video odometry based on the parametric representation of lanes include, e.g., encoding and/or decoding parametric representations into lane lines. In one embodiment, the geographic database 115 include high resolution or high definition (HD) mapping data that provide centimeter-level or better accuracy of map features. For example, the geographic database 115 can be based on Light Detection and Ranging (LiDAR) or equivalent technology to collect billions of 3D points and model road surfaces and other map features down to the number lanes and their widths. In one embodiment, the mapping data (e.g., mapping data records 811) capture and store details such as the slope and curvature of the road, lane markings, roadside objects such as signposts, including what the signage denotes. By way of example, the mapping data enable highly automated vehicles to precisely localize themselves on the road.

In one embodiment, geographic features (e.g., two-dimensional or three-dimensional features) are represented using polygons (e.g., two-dimensional features) or polygon extrusions (e.g., three-dimensional features). For example, the edges of the polygons correspond to the boundaries or edges of the respective geographic feature. In the case of a building, a two-dimensional polygon can be used to represent a footprint of the building, and a three-dimensional polygon extrusion can be used to represent the three-dimensional surfaces of the building. It is contemplated that although various embodiments are discussed with respect to two-dimensional polygons, it is contemplated that the embodiments are also applicable to three-dimensional polygon extrusions. Accordingly, the terms polygons and polygon extrusions as used herein can be used interchangeably.

In one embodiment, the following terminology applies to the representation of geographic features in the geographic database 115.

“Node”—A point that terminates a link.

“Line segment”—A straight line connecting two points.

“Link” (or “edge”)—A contiguous, non-branching string of one or more line segments terminating in a node at each end.

“Shape point”—A point along a link between two nodes (e.g., used to alter a shape of the link without defining new nodes).

“Oriented link”—A link that has a starting node (referred to as the “reference node”) and an ending node (referred to as the “non reference node”).

“Simple polygon”—An interior area of an outer boundary formed by a string of oriented links that begins and ends in one node. In one embodiment, a simple polygon does not cross itself.

“Polygon”—An area bounded by an outer boundary and none or at least one interior boundary (e.g., a hole or island). In one embodiment, a polygon is constructed from one outer simple polygon and none or at least one inner simple polygon. A polygon is simple if it just consists of one simple polygon, or complex if it has at least one inner simple polygon.

In one embodiment, the geographic database 115 follows certain conventions. For example, links do not cross themselves and do not cross each other except at a node. Also, there are no duplicated shape points, nodes, or links. Two links that connect each other have a common node. In the geographic database 115, overlapping geographic features are represented by overlapping polygons. When polygons overlap, the boundary of one polygon crosses the boundary of the other polygon. In the geographic database 115, the location at which the boundary of one polygon intersects they boundary of another polygon is represented by a node. In one embodiment, a node may be used to represent other locations along the boundary of a polygon than a location at which the boundary of the polygon intersects the boundary of another polygon. In one embodiment, a shape point is not used to represent a point at which the boundary of a polygon intersects the boundary of another polygon.

As shown, the geographic database 115 includes node data records 803, road segment or link data records 805, POI data records 807, vaccination data records 809, mapping data records 811, and indexes 813, for example. More, fewer or different data records can be provided. In one embodiment, additional data records (not shown) can include cartographic (“carto”) data records, routing data, and maneuver data. In one embodiment, the indexes 813 may improve the speed of data retrieval operations in the geographic database 115. In one embodiment, the indexes 813 may be used to quickly locate data without having to search every row in the geographic database 115 every time it is accessed. For example, in one embodiment, the indexes 813 can be a spatial index of the polygon points associated with stored feature polygons.

In exemplary embodiments, the road segment data records 805 are links or segments representing roads, streets, or paths, as can be used in the calculated route or recorded route information for determination of one or more personalized routes. The node data records 803 are end points corresponding to the respective links or segments of the road segment data records 805. The road link data records 805 and the node data records 803 represent a road network, such as used by vehicles, cars, and/or other entities. Alternatively, the geographic database 115 can contain path segment and node data records or other data that represent pedestrian paths or areas in addition to or instead of the vehicle road record data, for example.

The road/link segments and nodes can be associated with attributes, such as geographic coordinates, street names, address ranges, speed limits, turn restrictions at intersections, and other navigation related attributes, as well as POIs, such as gasoline stations, hotels, restaurants, museums, stadiums, offices, automobile dealerships, auto repair shops, buildings, stores, parks, etc. The geographic database 115 can include data about the POIs and their respective locations in the POI data records 807. The geographic database 115 can also include data about places, such as cities, towns, or other communities, and other geographic features, such as bodies of water, mountain ranges, etc. Such place or feature data can be part of the POI data records 807 or can be associated with POIs or POI data records 807 (such as a data point used for displaying or representing a position of a city).

In one embodiment, the geographic database 115 can also include vaccination data records 809 for storing static and/or dynamic vaccination data, training data, prediction models, annotated observations, computed vaccinated/unvaccinated population distribution data, sampling probabilities, and/or any other data generated or used by the system 100 according to the various embodiments described herein. By way of example, the vaccination data records 809 can be associated with one or more of the node records 803, road segment records 805, and/or POI data records 807 to support localization or visual odometry based on the features stored therein and the corresponding estimated quality of the features. In this way, the vaccination data records 809 can also be associated with or used to classify the characteristics or metadata of the corresponding records 803, 805, and/or 807.

In one embodiment, as discussed above, the mapping data records 811 model road surfaces and other map features to centimeter-level or better accuracy. The mapping data records 811 also include lane models that provide the precise lane geometry with lane boundaries, as well as rich attributes of the lane models. These rich attributes include, but are not limited to, lane traversal information, lane types, lane marking types, lane level speed limit information, and/or the like. In one embodiment, the mapping data records 811 are divided into spatial partitions of varying sizes to provide mapping data to vehicles 103 and other end user devices with near real-time speed without overloading the available resources of the vehicles 103 and/or devices (e.g., computational, memory, bandwidth, etc. resources).

In one embodiment, the mapping data records 811 are created from high-resolution 3D mesh or point-cloud data generated, for instance, from LiDAR-equipped vehicles. The 3D mesh or point-cloud data are processed to create 3D representations of a street or geographic environment at centimeter-level accuracy for storage in the mapping data records 811.

In one embodiment, the mapping data records 811 also include real-time sensor data collected from probe vehicles in the field. The real-time sensor data, for instance, integrates real-time traffic information, weather, and road conditions (e.g., potholes, road friction, road wear, etc.) with highly detailed 3D representations of street and geographic features to provide precise real-time also at centimeter-level accuracy. Other sensor data can include vehicle telemetry or operational data such as windshield wiper activation state, braking state, steering angle, accelerator position, and/or the like.

In one embodiment, the geographic database 115 can be maintained by the content providers 123 in association with the services platform 119 (e.g., a map developer). The map developer can collect geographic data to generate and enhance the geographic database 115. There can be different ways used by the map developer to collect data. These ways can include obtaining data from other sources, such as municipalities or respective geographic authorities. In addition, the map developer can employ field personnel to travel by vehicle (e.g., vehicles 103 and/or UE 101) along roads throughout the geographic region to observe features and/or record information about them, for example. Also, remote sensing, such as aerial or satellite photography, can be used.

The geographic database 115 can be a master geographic database stored in a format that facilitates updating, maintenance, and development. For example, the master geographic database or data in the master geographic database can be in an Oracle spatial format or other spatial format, such as for development or production purposes. The Oracle spatial format or development/production database can be compiled into a delivery format, such as a geographic data files (GDF) format. The data in the production and/or delivery formats can be compiled or further compiled to form geographic database products or databases, which can be used in end user navigation devices or systems.

For example, geographic data is compiled (such as into a platform specification format (PSF) format) to organize and/or configure the data for performing navigation-related functions and/or services, such as route calculation, route guidance, map display, speed calculation, distance and travel time functions, and other functions, by a navigation device, such as by a vehicle 103 or a UE 101, for example. The navigation-related functions can correspond to vehicle navigation, pedestrian navigation, or other types of navigation. The compilation to produce the end user databases can be performed by a party or entity separate from the map developer. For example, a customer of the map developer, such as a navigation device developer or other end user device developer, can perform compilation on a received geographic database in a delivery format to produce one or more compiled navigation databases.

The processes described herein for providing navigation and location recommendation based on geospatial vaccination data may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

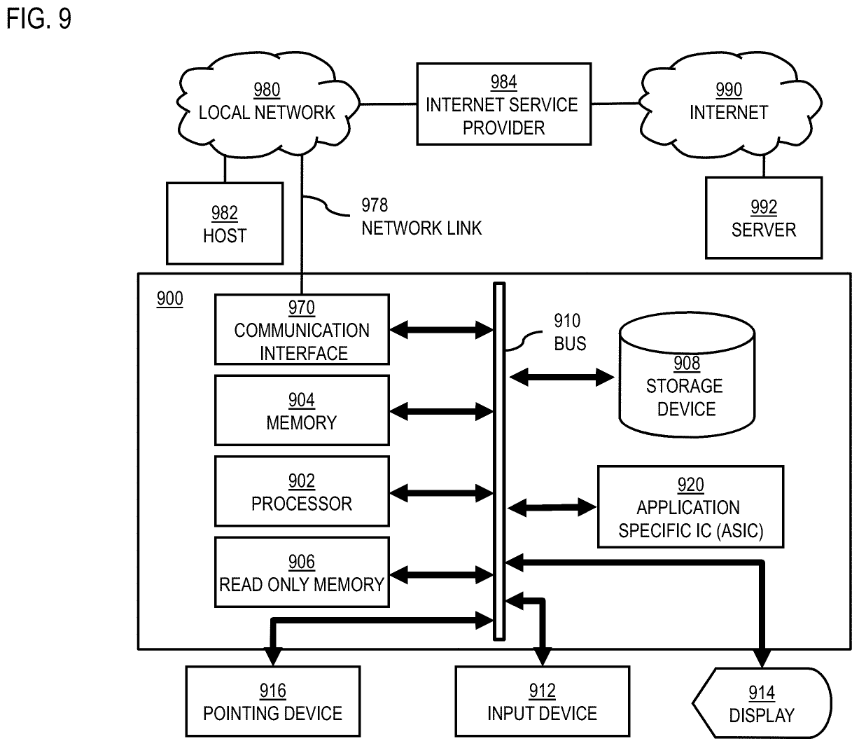

FIG. 9 illustrates a computer system 900 upon which an embodiment of the invention may be implemented. Computer system 900 is programmed (e.g., via computer program code or instructions) to provide navigation and location recommendation based on geospatial vaccination data as described herein and includes a communication mechanism such as a bus 910 for passing information between other internal and external components of the computer system 900. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 910 includes one or more parallel conductors of information so that information is transferred quickly among devices coupled to the bus 910. One or more processors 902 for processing information are coupled with the bus 910.

A processor 902 performs a set of operations on information as specified by computer program code related to providing navigation and location recommendation based on geospatial vaccination data. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 910 and placing information on the bus 910. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 902, such as a sequence of operation codes, constitute processor instructions, also called computer system instructions or, simply, computer instructions. The processors 902 may be implemented as mechanical, electrical, magnetic, optical, chemical or quantum components, among others, alone or in combination.

Computer system 900 also includes a memory 904 coupled to bus 910. The memory 904, such as a random access memory (RAM) or other dynamic storage device, stores information including processor instructions for providing navigation and location recommendation based on geospatial vaccination data. Dynamic memory allows information stored therein to be changed by the computer system 900. RAM allows a unit of information stored at a location called a memory address to be stored and retrieved independently of information at neighboring addresses. The memory 904 is also used by the processor 902 to store temporary values during execution of processor instructions. The computer system 900 also includes a read only memory (ROM) 906 or other static storage device coupled to the bus 910 for storing static information, including instructions, that is not changed by the computer system 900. Some memory is composed of volatile storage that loses the information stored thereon when power is lost. Also coupled to bus 910 is a non-volatile (persistent) storage device 908, such as a magnetic disk, optical disk or flash card, for storing information, including instructions, that persists even when the computer system 900 is turned off or otherwise loses power.

Information, including instructions for providing navigation and location recommendation based on geospatial vaccination data, is provided to the bus 910 for use by the processor from an external input device 912, such as a keyboard containing alphanumeric keys operated by a human user, or a sensor. A sensor detects conditions in its vicinity and transforms those detections into physical expression compatible with the measurable phenomenon used to represent information in computer system 900. Other external devices coupled to bus 910, used primarily for interacting with humans, include a display device 914, such as a cathode ray tube (CRT) or a liquid crystal display (LCD), or plasma screen or printer for presenting text or images, and a pointing device 916, such as a mouse or a trackball or cursor direction keys, or motion sensor, for controlling a position of a small cursor image presented on the display 914 and issuing commands associated with graphical elements presented on the display 914. In some embodiments, for example, in embodiments in which the computer system 900 performs all functions automatically without human input, one or more of external input device 912, display device 914 and pointing device 916 is omitted.

In the illustrated embodiment, special purpose hardware, such as an application specific integrated circuit (ASIC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a one-way or two-way communication coupling to a variety of external devices that operate with their own processors, such as printers, scanners and external disks. In general the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. For wireless links, the communications interface 970 sends or receives or both sends and receives electrical, acoustic or electromagnetic signals, including infrared and optical signals, that carry information streams, such as digital data. For example, in wireless handheld devices, such as mobile telephones like cell phones, the communications interface 970 includes a radio band electromagnetic transmitter and receiver called a radio transceiver. In certain embodiments, the communications interface 970 enables connection to the communication network 117 for providing navigation and location recommendation based on geospatial vaccination data.

The term computer-readable medium is used herein to refer to any medium that participates in providing information to processor 902, including instructions for execution. Such a medium may take many forms, including, but not limited to, non-volatile media, volatile media and transmission media. Non-volatile media include, for example, optical or magnetic disks, such as storage device 908. Volatile media include, for example, dynamic memory 904. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, any other memory chip or cartridge, a carrier wave, or any other medium from which a computer can read.

FIG. 10 illustrates a chip set 1000 upon which an embodiment of the invention may be implemented. Chip set 1000 is programmed to provide navigation and location recommendation based on geospatial vaccination data as described herein and includes, for instance, the processor and memory components described with respect to FIG. 9 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform the inventive steps described herein to provide navigation and location recommendation based on geospatial vaccination data. The memory 1005 also stores the data associated with or generated by the execution of the inventive steps.

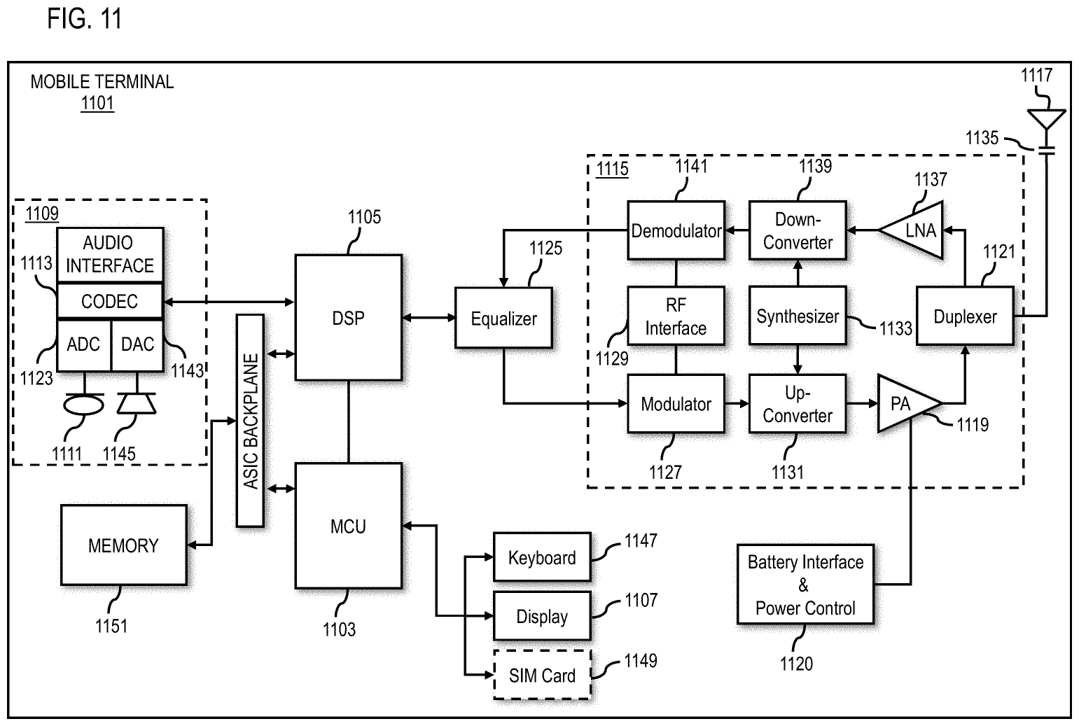

FIG. 11 is a diagram of exemplary components of a mobile terminal 1101 (e.g., a mobile device, a vehicle or part thereof, or client device such as the UE 101) capable of operating in the system of FIG. 1, according to one embodiment. Generally, a radio receiver is often defined in terms of front-end and back-end characteristics. The front-end of the receiver encompasses all of the Radio Frequency (RF) circuitry whereas the back-end encompasses all of the base-band processing circuitry. Pertinent internal components of the telephone include a Main Control Unit (MCU) 1103, a Digital Signal Processor (DSP) 1105, and a receiver/transmitter unit including a microphone gain control unit and a speaker gain control unit. A main display unit 1107 provides a display to the user in support of various applications and mobile terminal functions that offer automatic contact matching. An audio function circuitry 1109 includes a microphone 1111 and microphone amplifier that amplifies the speech signal output from the microphone 1111. The amplified speech signal output from the microphone 1111 is fed to a coder/decoder (CODEC) 1113.

A radio section 1115 amplifies power and converts frequency in order to communicate with a base station, which is included in a mobile communication system, via antenna 1117. The power amplifier (PA) 1119 and the transmitter/modulation circuitry are operationally responsive to the MCU 1103, with an output from the PA 1119 coupled to the duplexer 1121 or circulator or antenna switch, as known in the art. The PA 1119 also couples to a battery interface and power control unit 1120.

In use, a user of mobile terminal 1101 speaks into the microphone 1111 and his or her voice along with any detected background noise is converted into an analog voltage. The analog voltage is then converted into a digital signal through the Analog to Digital Converter (ADC) 1123. The control unit 1103 routes the digital signal into the DSP 1105 for processing therein, such as speech encoding, channel encoding, encrypting, and interleaving. In one embodiment, the processed voice signals are encoded, by units not separately shown, using a cellular transmission protocol such as global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wireless fidelity (WiFi), satellite, and the like.

The encoded signals are then routed to an equalizer 1125 for compensation of any frequency-dependent impairments that occur during transmission though the air such as phase and amplitude distortion. After equalizing the bit stream, the modulator 1127 combines the signal with a RF signal generated in the RF interface 1129. The modulator 1127 generates a sine wave by way of frequency or phase modulation. In order to prepare the signal for transmission, an up-converter 1131 combines the sine wave output from the modulator 1127 with another sine wave generated by a synthesizer 1133 to achieve the desired frequency of transmission. The signal is then sent through a PA 1119 to increase the signal to an appropriate power level. In practical systems, the PA 1119 acts as a variable gain amplifier whose gain is controlled by the DSP 1105 from information received from a network base station. The signal is then filtered within the duplexer 1121 and optionally sent to an antenna coupler 1135 to match impedances to provide maximum power transfer. Finally, the signal is transmitted via antenna 1117 to a local base station. An automatic gain control (AGC) can be supplied to control the gain of the final stages of the receiver. The signals may be forwarded from there to a remote telephone which may be another cellular telephone, other mobile phone or a land-line connected to a Public Switched Telephone Network (PSTN), or other telephony networks.

Voice signals transmitted to the mobile terminal 1101 are received via antenna 1117 and immediately amplified by a low noise amplifier (LNA) 1137. A down-converter 1139 lowers the carrier frequency while the demodulator 1141 strips away the RF leaving only a digital bit stream. The signal then goes through the equalizer 1125 and is processed by the DSP 1105. A Digital to Analog Converter (DAC) 1143 converts the signal and the resulting output is transmitted to the user through the speaker 1145, all under control of a Main Control Unit (MCU) 1103—which can be implemented as a Central Processing Unit (CPU) (not shown).

The MCU 1103 receives various signals including input signals from the keyboard 1147. The keyboard 1147 and/or the MCU 1103 in combination with other user input components (e.g., the microphone 1111) comprise a user interface circuitry for managing user input. The MCU 1103 runs a user interface software to facilitate user control of at least some functions of the mobile terminal 1101 to provide navigation and location recommendation based on geospatial vaccination data. The MCU 1103 also delivers a display command and a switch command to the display 1107 and to the speech output switching controller, respectively. Further, the MCU 1103 exchanges information with the DSP 1105 and can access an optionally incorporated SIM card 1149 and a memory 1151. In addition, the MCU 1103 executes various control functions required of the mobile station 1101. The DSP 1105 may, depending upon the implementation, perform any of a variety of conventional digital processing functions on the voice signals. Additionally, DSP 1105 determines the background noise level of the local environment from the signals detected by microphone 1111 and sets the gain of microphone 1111 to a level selected to compensate for the natural tendency of the user of the mobile terminal 1101.

The CODEC 1113 includes the ADC 1123 and DAC 1143. The memory 1151 stores various data including call incoming tone data and is capable of storing other data including music data received via, e.g., the global Internet. The software module could reside in RAM memory, flash memory, registers, or any other form of writable computer-readable storage medium known in the art including non-transitory computer-readable storage medium. For example, the memory device 1151 may be, but not limited to, a single memory, CD, DVD, ROM, RAM, EEPROM, optical storage, or any other non-volatile or non-transitory storage medium capable of storing digital data.

An optionally incorporated SIM card 1149 carries, for instance, important information, such as the cellular phone number, the carrier supplying service, subscription details, and security information. The SIM card 1149 serves primarily to identify the mobile terminal 1101 on a radio network. The card 1149 also contains a memory for storing a personal telephone number registry, text messages, and user specific mobile terminal settings.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. A method comprising:
initiating a query of digital map data for vaccination data associated with a geographic area, wherein the vaccination data indicates a geospatial density of a vaccinated population in the geographic area;
processing the vaccination data to determine a navigation route, a recommended point of interest, a recommended event plan, or a combination thereof in the geographic area based on the vaccination data; and
providing the navigation route, the recommended point of interest, the recommended event plan, or a combination thereof as an output.

2. The method of claim 1, further comprising:
receiving a request specifying or providing data for specifying a threshold density of the vaccinated population,
wherein the navigation route, the recommended point of interest, the recommended event plan, or a combination thereof is determined further based on the threshold density.

3. The method of claim 2, wherein the navigation route, the recommended point of interest, the recommended event plan, or a combination thereof is determined based favoring or avoiding one or more portions of the geographic area associated with vaccination data indicating that the vaccinated population is above or below the threshold density.

4. The method of claim 2, wherein the data for specifying the threshold density includes user medical condition data regarding at least one user associated with the request, and wherein the threshold density is determined based on the user medical condition data.

5. The method of claim 4, wherein the user medical condition data includes a vaccination status of the at least one user.

6. The method of claim 2, wherein the request further specifies a travel time, a travel distance, or a combination thereof, and wherein the navigation route, the recommended point of interest, the recommended event plan, or a combination thereof is determined further based on the travel time, the travel distance, or a combination thereof.

7. The method of claim 1, further comprising:
monitoring one or more real-time parameters that affect the geospatial density of the vaccinated population; and
updating the navigation route, the recommended point of interest, the recommended event plan, or a combination thereof based on the one or more real-time parameters.

8. The method of claim 1, wherein the vaccinated population includes a working population working at the recommended point of interest, at a location associated with the recommended event plan, within a threshold distance of the navigation route, or a combination thereof.

9. The method of claim 1, wherein the navigation route, the recommended point of interest, the recommended event plan, or a combination thereof relates to indoor navigation or indoor mapping.

10. The method of claim 1, further comprising:
processing the output to generate a map user interface presenting a real-time map representation of the vaccination data with respect to the geographic area, the navigation route, the recommended point of interest, the recommended event plan, or a combination.

11. An apparatus comprising:
at least one processor; and
at least one memory including computer program code for one or more programs,
the at least one memory and the computer program code configured to, with the at least one processor, cause the apparatus to perform at least the following,
monitor a vaccination database for vaccination status data for one or more individuals;
initiate a correlation of the vaccination status data, the one or more individuals, or a combination thereof to one or more locations in a geographic area;
process the correlation to determine a geospatial density of a vaccinated population in the geographic area;
store the geospatial density of the vaccinated population as vaccination data of a geographic database; and
provide the geographic database as an output,
wherein a navigation route, a recommended point of interest, a recommended event plan, or a combination thereof is determined based on the vaccination data of the geographic database.

12. The apparatus of claim 11, wherein the apparatus is further caused to:
anonymize the vaccination status data for the one or more individuals,
wherein the vaccination data of the geographic database is based on the anonymized vaccination status data.

13. The apparatus of claim 11, wherein the apparatus is further caused to:
collect mobility data from one or more devices associated with the one or more individuals,
wherein the geospatial density of the vaccinated population is determined further based on the mobility data.

14. The apparatus of claim 11, wherein the vaccination status data, the mobility data, or a combination thereof is crowd-sourced from the one or more individuals who consent to collecting the vaccination status data, the mobility data, or combination thereof.

15. The apparatus of claim 11, further comprising:
providing access to the geographic database to at least one edge device,
wherein the edge device submits at least one request to generate the navigation route, the recommended point of interest, the recommended event plan, or a combination thereof.

16. A non-transitory computer-readable storage medium carrying one or more sequences of one or more instructions which, when executed by one or more processors, cause an apparatus to perform:
receiving a destination as input;
determining a route from a current location to the destination via a plurality of road segments, wherein one or more of the plurality of road segments to be part of the route is determined based on vaccination data associated with the one or more of the plurality of road segments; and
outputting the determined route or a portion thereof.

17. The non-transitory computer-readable storage medium of claim 16, wherein the vaccination data includes one or more markers or indicators associating a person, a location, or a combination thereof with vaccination.

18. The non-transitory computer-readable storage medium of claim 17, wherein the apparatus is caused to further perform:
receiving, via the user interface, a request specifying a threshold density of the vaccinated data,
wherein the determined route or the portion thereof is determined further based on the threshold density.

19. The non-transitory computer-readable storage medium of claim 18, wherein the apparatus is caused to further perform:
receiving, at the device, digital map data including the vaccination data,
wherein the determined route or the portion thereof is determined based favoring or avoiding the plurality of road segments associated with vaccination data indicating that vaccinated population is above or below the threshold density.

20. The non-transitory computer-readable storage medium of claim 16, wherein the apparatus is caused to further perform:
receiving, via the user interface, a user consent to share user vaccination status data, user mobility data, or a combination thereof,
wherein the vaccination data, the determined route or the portion thereof, or a combination thereof is updated further based on the user vaccination status data, the user mobility data, or a combination thereof.
